(19) United States
(12) Patent Application Publication
(10) Pub. No.: US 2026/0326986 A1
(43) Pub. Date: Oct. 22, 2026

(54) STOPPER FOR RELEASING A SUBSTANCE, ASSOCIATED KIT AND ACTUATION METHOD

(71) Applicant: COPAN ITALIA S.p.A., BRESCIA (IT)

(72) Inventors: Giorgio MARTELLO, BRESCIA (IT); Matteo ZANI, BRESCIA (IT)

(21) Appl. No.: 19/114,166

(22) PCT Filed: Sep. 6, 2023
(86) PCT No.: PCT/IB2023/058803

§ 371 Date: Mar. 21, 2025

(30) Foreign Application Priority Data
Sep. 22, 2022 (IT) 102022000019494

(51) Int. Cl. B65D 51/28; B65D 39/00

(52) U.S. Cl. CPC B65D 51/2835; B65D 39/00

(57) ABSTRACT

Stopper (1) for releasing a substance (2) into a container (3) for samples, comprising:
a first body (4, 5) engaged on said container (3),
a second body (7) movable with respect to the first body (4, 5),
a tank (6) containing the substance (2) and defined between the first body (4, 5) and the second body (7);
   wherein the first body (4, 5) comprises:
one first portion (4) comprising a coupling element (41) allowing the fastening of the stopper (1) with the container (3) defining a first mounting configuration of the stopper on the container,
one second portion (5) connected with said first portion (4) and realizing part of a bottom wall of said tank (6), said second portion (5) being arranged at one first position with respect to said first portion (4) or at one second position with respect to said first portion (4);
   said second portion (5) being coupled to said second body (7);
   the tank (6) having an open configuration and a closed configuration, said open configuration being determined by the first position between the first portion (4) and the second portion (5), the closed configuration being determined by the second position between the first portion (4) and the second portion (5).

### TECHNICAL FIELD

The present disclosure refers to the field of closing elements for containers and in detail concerns a stopper for releasing a substance. The present disclosure also illustrates a kit comprising the stopper and a device for taking samples, preferably for collecting samples of biological and/or microbiological material, and/or comprising a container for samples, preferably for samples of biological and/or microbiological material.

### BACKGROUND ART

The stoppers for releasing substances are stoppers configured to be removably coupled with containers and also configured to store substances in liquid or solid form, for example in powder form, in order to release it under determined conditions, for example within a container with which the stopper is associated. Stoppers of known type typically comprise a first operating configuration in which the substance is contained in a tank of defined volume within the stopper and a second operating configuration in which the tank is opened, allowing the release of the substance. Typically the release of the substance occurs within the container in a configuration wherein the stopper is constrained to the container.

Stoppers of known type can present a movable part, typically not the one directly connected to the container itself, which has breaking elements capable of opening a membrane that puts the recess in communication with the outer environment.

These stoppers can be opened inadvertently and release the substance. The opening can occur both with the stopper properly fixed to the container and with the stopper free of the container. If the substance is harmful, or likely to degrade, the release of the substance outside the container may be dangerous or lead to degradation and consequent substantial unusability of the substance.

It should be noted that stoppers of known type are often used not only by professional operators but also by private users, and can be handled by children.

In the field of collecting and/or storing and/or analyzing biological and microbiological samples, it is known the use of test tubes or more generally of containers, closable by a stopper, within which to contain biological samples, either loose or deposited through a device for collecting samples.

It is also known that samples, and in particular samples of biological material may have to be subjected to treatment with reagents that may be poisonous or anyway toxic.

In determined types of analysis, the use of the reagent must be subject to the use in a closed environment, i.e. with direct release within the container, for reasons of chemical reaction, degradation of the reagent, or operator safety. Furthermore, some reagents are very expensive, and their dispersion—further than risking to cause the above-mentioned issues—can also involve a considerable expense.

From patent application US 2013/0092690 it is known a container for biological sample provided with a stopper capable of containing an agent; the stopper presents an ampoule and a perforating element moving within the housing of the stopper. If the stopper is mishandled, and in particular if it is compressed along its longitudinal axis, the content of the ampoule may be inadvertently let out, with the consequent risk of exposure of the operator to danger and/or with the consequent inevitable dispersion of the agent contained in the ampoule.

It is observed that this section is presented solely for the purpose of showing certain issues that the object of the invention is intended to address. The embodiments eventually described in this section are not to be intended as part of the state of the art merely because they have been described herein.

It has also been noted that many stoppers for releasing substances are provided with structures comprising a large number of disjointed parts provided with sealing rings among them, which contribute to cause a constructive and assembly complexity of the stopper itself. Such sealing rings are in particular realized with silicone or rubbery material which can lose its plastic deformability capabilities over time, with the consequent risk of leakage of the substance contained in the stopper. Furthermore, such silicone or rubbery material may not be chemically compatible, or anyway react, with certain substances or reagents present in the tank of the stopper. The Applicant has in fact observed that stoppers containing substances are often stored for long periods, even longer than a year.

The purpose of the present disclosure is to describe a stopper for releasing substances which is capable of solving the above-described drawbacks.

The purpose of the present disclosure is also to describe a kit comprising said stopper, for solving the above-described drawbacks.

The purpose of the present disclosure is to describe a method of actuation of the stopper, for solving the above-described drawbacks.

The purpose of the present disclosure is also to describe a machine for handling the stopper described herein.

### SUMMARY

The object of the present disclosure is herein described in some of its salient aspects. These aspects may be combined with each other and/or with portions of the detailed description and/or claims.

It should be noted that in the following aspects, expressions such as “in particular” or “preferably” are intended to be non-limiting.

### Stopper

According to the present disclosure it is described a stopper (1) for releasing a substance (2) into a container (3) for samples, comprising at least:
a first body (4, 5) configured to be removably engaged on said container (3),
a second body (7) coupled and movable with respect to the first body (4, 5),
at least one tank (6) configured to contain the substance (2) and defined in the first body (4, 5) or between the first body (4, 5) and the second body (7);
   wherein the first body (4, 5) comprises at least:
a first portion (4) configured to be directly coupled on said container (3) and comprising a coupling element (41) configured to allow the removable fastening of the stopper (1) in correspondence of an opening of the container (3) defining a first mounting configuration of the stopper on the container wherein, preferably, the stopper (1) closes the container (3),
a second portion (5) movably connected with said first portion (4), said second portion (5) realizing at least part of a bottom wall and/or of a lateral wall of said tank (6), said second portion (5) being capable to lie in at least one first relative position with respect to said first portion (4) and in at least one second relative position with respect to said first portion (4);
   said second portion (5) of the first body (4, 5) being directly coupled to said second body (7),
   the tank (6) having at least one first open configuration and a second closed configuration, said first open configuration being determined by the first relative position between the first portion (4) of the first body (4, 5) and the second portion (5) of the first body (4, 5), said second closed configuration being determined by the second relative position between the first portion (4) of the first body (4, 5) and the second portion (5) of the first body (4, 5).

According to a further non-limiting aspect, said container (3) is a container for samples of biological and/or microbiological material.

According to a further non-limiting aspect, the tank (6) is a tank having a volume variable at least between a first volume and a second volume, the second volume being smaller with respect to the first volume.

According to a further non-limiting aspect, the second body (7) is configured to assume at least one first position and one second position with respect to said first body (4, 5).

According to a further non-limiting aspect, a movement, in use, of the second body (7) with respect to the first body (4, 5) between said first position and said second position determines a variation of volume of said tank (6) and/or the tank (6) assumes the first volume when said second body (7) is in said first position with respect to said first body (4, 5) and the tank (6) assumes the second volume when said second body (7) is in said second position with respect to said first body (4, 5),
optionally wherein the second body (7) is movable with respect to the first body (4, 5) between said first position and said second position through at least one axial translation.

According to a further non-limiting aspect, said first position and said second position are end stroke positions for said axial translation, and said first volume is a maximum volume and/or said second volume is a minimum volume.

According to a further non-limiting aspect, the stopper (1) presents a main development axis (X) and said axial translation occurs along a parallel direction, preferably along a direction substantially coincident, with said main development axis (X).

According to a further non-limiting aspect, the stopper comprises at least one discharge hole (9) for allowing to discharge the substance (2) from said tank (6).

According to a further non-limiting aspect, said at least one discharge hole (9) is arranged in said first portion (4) of the first body (4, 5).

According to a further non-limiting aspect, said second portion (5) of the first body (4, 5) comprises at least one closing element (10) configured to removably engage within said at least one discharge hole (9) and comprises at least one through hole (54) configured to couple with said discharge hole (9) alternatively to said at least one closing element (10), or said at least one discharge hole (9) is arranged in said second portion (5) of the first body (4, 5) and said first portion (4) of the first body (4, 5) comprises at least one closing element (10) configured to removably engage within said at least one discharge hole (9) and comprises at least one through hole (54) configured to couple with said discharge hole (9) alternatively to said closing element (10),

preferably when the first portion (4) of the first body (4, 5) is in said second relative position with respect to the second portion (5) of the first body (4, 5), the closing element (10) is positioned at least partially, preferably entirely, within said discharge hole (9) and/or seals said discharge hole (9) and the through hole (54) is uncoupled with respect to said discharge hole (9), and when the first portion (4) of the first body (4, 5) is in said first relative position with respect to the second portion (5) the closing element (10) is positioned at least partially, preferably entirely, outside of said discharge hole (9) and/or frees at least partially, preferably entirely, said discharge hole (9), and the through hole (54) is coupled to said discharge hole (9),
optionally wherein when the first portion (4) of the first body (4, 5) is in said second relative position with respect to the second portion (5) of the first body (4, 5), said at least one discharge hole (9) is insulated with respect to said tank (6) and when the first portion (4) of the first body (4, 5) is in said first relative position with respect to the second portion (5) of the first body (4, 5), said at least one discharge hole (9) communicates with said tank (6).

According to a further non-limiting aspect, the stopper (1) is configured to be removably coupled with a rod (100) of a device (100, 101) for collecting samples, and preferably to be removably coupled with a rod (100) of a device (100, 101) for collecting samples of biological and/or microbiological material.

According to a further non-limiting aspect, the device (100, 101) for collecting samples comprises a swab (101), joined with, at least partially superimposed on, realized on, said rod (100) and configured and specifically destined to allow the collection and/or the storage of a sample, preferably destined to allow the collection and/or the storage of a sample of biological and/or microbiological material.

According to a further non-limiting aspect, said swab (101) is a flocked swab.

According to a further non-limiting aspect, at least a part of the rod (100) is realized in plastic material, optionally being realized as one piece and/or being realized through a moulding process, in particular injection moulding.

According to a further non-limiting aspect, the first portion (4) of the first body (4, 5) comprises at least one cavity (8c) realizing a housing configured to retain removably, in use, an end portion of said rod (100).

According to a further non-limiting aspect, the first portion (4) of the first body (4, 5) comprises at least one channel (4v), communicating with said at least one discharge hole (9).

According to a further non-limiting aspect, said at least one channel (4v) is configured and specifically destined to allow an evacuation of the substance (2) extracted from the tank (6) in substantial proximity of the rod (100).

According to a further non-limiting aspect, said channel (4v) is communicating with, or is part of, said cavity (8c).

According to a further non-limiting aspect, the cavity (8c) is configured to allow an introduction of the end portion of said rod (100) through an axial insertion.

According to a further non-limiting aspect, the cavity (8c) is configured to allow an axial insertion of the end portion of said rod (100) along an axis parallel or coinciding with a main development axis (X) of said stopper (1).

According to a further non-limiting aspect, the first portion (4) of the first body (4, 5) comprises, in correspondence of said cavity (8c), at least one retaining rib (8n), preferably a plurality of retaining ribs (8n).

According to a further non-limiting aspect, said plurality of retaining ribs (8n) comprises at least one pair of retaining ribs (8n) and more preferably at least four retaining ribs (8n).

According to a further non-limiting aspect, said at least one pair of retaining ribs (8n) comprises at least one first and one second rib opposed to each other and/or radially opposed with respect to the development axis of the first portion (4) of the first body (4, 5).

According to a further non-limiting aspect, said at least one retaining rib (8n) radially protrudes towards a centre of said cavity (8c), and is destined to exert a sliding friction with said end portion of said rod (100) when introduced within said cavity (8c).

According to a further non-limiting aspect, the at least one retaining rib (8n) is elastically deformable.

According to a further non-limiting aspect, said plurality of retaining ribs (8n) realizes said at least one channel (4v) and/or said at least one channel (4v) is arranged between two contiguous retaining ribs (8n).

According to a further non-limiting aspect, the cavity (8c) is configured to exert a sliding friction with said end portion of said rod (100).

According to a further non-limiting aspect, said sliding friction is sufficient to determine a retention of the rod (100) with greater force with respect to the weight of the device for collecting samples.

According to a further non-limiting aspect, said at least one retaining rib (8n) comprises a radially outer portion and a radially inner portion.

According to a further non-limiting aspect, the radially inner portion is pointed.

According to a further non-limiting aspect, the plurality of radially pointed inner portions of the plurality of retaining ribs (8n) identifies, as a whole, a shape substantially corresponding to the shape of the end portion section of the rod (100).

According to a further non-limiting aspect, said shape has substantially an area substantially equal to or smaller than the area identified by the section of the end portion of the rod (100).

According to a further non-limiting aspect, the second portion (5) of the first body (4, 5) is rotatably connected with respect to said first portion (4) of the first body (4, 5).

According to a further non-limiting aspect, between said first relative position and said second relative position there is a predetermined rotation between said first portion (4) of the first body (4, 5) and said second portion (5) of the first body (4, 5).

According to a further non-limiting aspect, the second portion (5) of the first body (4, 5) is rotatably connected with respect to said first portion (4) of the first body (4, 5) and is configured to rotate with respect to said first portion (4) of the first body (4, 5) and on an axis substantially coinciding with a main development axis (X) of said stopper (1) and/or the second portion (5) of the first body (4, 5) is rotatably connected with respect to said first portion (4) of the first body (4, 5) on an axis substantially coinciding with a translation axis of the second body (7) with respect to the first body (4, 5).

According to a further non-limiting aspect, the translation axis of the second body (7) with respect to the first body (4, 5) is substantially coinciding with the main development axis (X) of said stopper (1).

According to a further non-limiting aspect, at least one between said first portion (4) of the first body (4, 5) and/or said second portion (5) of the first body (4, 5) comprises a respective dividing wall (4p, 5p) which is deformable, preferably elastically deformable.

According to a further non-limiting aspect, said dividing wall (4p, 5p) is at least partially, preferably substantially entirely, arranged in a direction orthogonal with respect to the main development axis (X) and/or is arranged in a direction orthogonal with respect to the translation axis of the second body (7) with respect to the first body (4, 5).

According to a further non-limiting aspect, a movement between the first portion (4) of the first body (4, 5) and the second portion (5) of the first body (4, 5) between said first relative position and said second relative position determines, in use, a deformation, preferably an elastic deformation, of at least one between the dividing wall (4p) of the first portion (4) and the dividing wall (5p) of the second portion (5) of the first body (4, 5).

According to a further non-limiting aspect, the dividing wall (5p) of the second portion (5) of the first body (4, 5) realizes a bottom wall of said tank (6).

According to a further non-limiting aspect, alternatively the at least one discharge hole (9) is realized on the dividing wall (4p) of the first portion (4) of the first body (4, 5) and the at least one through hole (54) and the at least one closing element (10) are arranged on the dividing wall (5p) of the second portion (5) of the first body (4, 5) or wherein the at least one discharge hole (9) is realized on the dividing wall (5p) of the second portion (5) of the first body (4, 5) and the at least one through hole (54) and the at least one closing element (10) are arranged on the dividing wall (4p) of the first portion (4) of the first body (4, 5).

According to a further non-limiting aspect, the first portion (4) of the first body (4, 5), comprises at least one first recess (11) within which the at least one closing element (10) slides during the relative movement, optionally the rotation, between the first portion (4) and the second portion (5) of the first body (4, 5).

According to a further non-limiting aspect, the second portion (5) of the first body (4, 5) comprises at least one first recess (11) within which the at least one closing element (10) slides during the relative movement, optionally the rotation, between the first portion (4) and the second portion (5) of the first body (4, 5).

According to a further non-limiting aspect, said recess (11) is a recess substantially identifying a curved profile, optionally a circumference arc and/or houses the at least one discharge hole (9).

According to a further non-limiting aspect, the at least one discharge hole (9) lies in substantial correspondence of a first end of the at least one first recess (11).

According to a further non-limiting aspect, the at least one first recess (11) is arranged on said dividing wall (4p) of the first portion (4) of the first body (4, 5).

According to a further non-limiting aspect, the at least one first recess (11) is arranged on said dividing wall (5p) of the second portion (5) of the first body (4, 5).

According to a further non-limiting aspect, the dividing wall (4p) of the first portion (4) comprises a first and a second recess (11), preferably opposed to each other, wherein the first closing element (10) is configured to slide in the first recess (11) and the second closing element (10) is configured to slide in the second recess (11) during the relative movement, optionally the rotation, between the first portion (4) and the second portion (5).

According to a further non-limiting aspect, the dividing wall (5p) of the second portion (5) comprises a first and a second recess (11), preferably opposed to each other, wherein the first closing element (10) is configured to slide in the first recess (11) and the second closing element (10) is configured to slide in the second recess (11) during the relative movement, optionally the rotation, between the first portion (4) and the second portion (5).

According to a further non-limiting aspect, said first and second recess (11) are recesses substantially identifying a curved profile, optionally a circumference arc and/or house, each one, a respective discharge hole (9).

According to a further non-limiting aspect, the at least one discharge hole (9) lies in substantial correspondence of a first end of the at least one recess (11).

According to a further non-limiting aspect, the first and the second recess (11) lie at a substantially same distance with respect to a center of said dividing wall (4p) of the first portion (4).

According to a further non-limiting aspect, said dividing wall (4p, 5p) of said first portion (4) and/or of said second portion (5) are substantially central walls of said first portion (4) and/or of said second portion (5) and/or axially aligned on said main development axis (X) of said stopper (1).

According to a further non-limiting aspect, at least one between said dividing wall (4p, 5p) of said first portion (4) and/or of said second portion (5) lies on a plane substantially orthogonal with respect to said main development axis (X) of said stopper (1).

According to a further non-limiting aspect, the stopper (1) is configured to allow a movement from the first relative position toward the second relative position between the first portion (4) of the first body (4, 5) and the second portion (5) of the first body (4, 5) through an initial snap and/or through an initial imposition of force greater with respect to a force required to complete the movement from said first relative position toward said second relative position.

According to a further non-limiting aspect, an extraction of said at least one closing element (10) from said at least one discharge hole (9) is made possible by said initial snap and/or by means of said initial imposition of force greater with respect to a force required to complete the movement from said first relative position toward said second relative position.

According to a further non-limiting aspect, said stopper (1) comprises at least one shoulder (5k) positioned in substantial correspondence of said at least one discharge hole (9).

According to a further non-limiting aspect, said shoulder has preferably an at least partially squared profile.

According to a further non-limiting aspect, said shoulder is configured and specifically destined to determine said initial snap and/or the initial imposition of said force greater with respect to a force required to complete the movement from said first relative position toward said second relative position.

According to a further non-limiting aspect, said at least one recess (11) comprises a connection portion (11r) with said at least one discharge hole (9).

According to a further non-limiting aspect, said connection portion (11r) presents a sliding surface for the closing element (10).

According to a further non-limiting aspect, said sliding surface is curved and, preferably, free of angular points.

According to a further non-limiting aspect, said connection portion (11r) is configured and specifically destined to facilitate the leakage of the closing element (10) from the respective discharge hole (9), optionally after said snap and/or said initial imposition of said force greater with respect to a force required to complete the movement from said first relative position toward said second relative position.

According to a further non-limiting aspect, said connection portion (11r) is configured and specifically destined to determine a variation of height assumed by the closing element (10), optionally along said main development axis (X) of the stopper (1), during the movement of the first portion (4) of the first body (4, 5) with respect to said second portion (5) of the first body (4, 5) between said first relative position and said second relative position.

According to a further non-limiting aspect, said at least one recess (11) has a substantially elongated shape and/or has at least one main development direction, and defines a first end stroke position and a second end stroke position for the relative movement, preferably the relative rotation, between the first portion (4) and the second portion (5) of the first body (4, 5).

According to a further non-limiting aspect, the at least one first recess (11) comprises a first end and a second end, and the first end stroke position is determined when the at least one closing element (10) is in correspondence of the first end of the at least one first recess (11).

According to a further non-limiting aspect, the second end stroke position is determined when the at least one closing element (10) is in correspondence of the second end of the at least one first recess (11).

According to a further non-limiting aspect, the stopper (1) comprises at least one safety or engagement element (11s), configured to allow a movement of the first portion (4) of the first body (4, 5) with respect to said second portion (5) of the first body (4, 5) from said first relative position toward said second relative position and to prevent a movement, in particular a backward movement, from said second relative position to said first relative position.

According to a further non-limiting aspect, said safety or engagement element (11s) is preferably configured to allow a rotation of the second portion (5) of the first body (4, 5) with respect to the first portion (4) of the first body (4, 5) in a first sense of rotation and to prevent, upon reaching a predefined position of rotation between said second portion (5) of the first body (4, 5) and said first portion (4) of the first body (4, 5), a rotation of said second portion (5) of the first body (4, 5) with respect to the first portion (4) of the first body (4, 5) in a second sense of rotation opposed with respect to said first sense of rotation.

According to a further non-limiting aspect, said safety or engagement element (11s) is realized in correspondence of said at least one first recess (11).

According to a further non-limiting aspect, said safety or engagement element (11s) is an anti-counterrotation element and/or comprises at least one blind hole arranged in correspondence of at least said first recess (11).

According to a further non-limiting aspect, said predefined position of rotation corresponds to said second end stroke position and/or to a predefined end of said at least one first recess (11).

According to a further non-limiting aspect, said predefined end is said second end.

According to a further non-limiting aspect, said safety or engagement element (11s) comprises an undercut or blind hole in correspondence of at least said first recess (11).

According to a further non-limiting aspect, said undercut or blind hole is positioned in correspondence of a bottom wall of said at least first recess (11).

According to a further non-limiting aspect, the second portion (5) of the first body (4, 5) comprises a lateral wall (52) delimiting at least part of said tank (6), and wherein the second body (7) is at least partially introduced in the second portion (5) of the first body (4, 5) and contacts said lateral wall (52).

According to a further non-limiting aspect, the second body (7) comprises a sealing element (71) in use in direct contact with said lateral wall (52).

According to a further non-limiting aspect, said sealing element (71) is deformable, in particular elastically deformable, and is deformed when the second body (7) is at least partially introduced in the second portion (5) of the first body (4, 5) and contacts said lateral wall (52).

According to a further non-limiting aspect, the second body (7) comprises a bottom wall (72) substantially joined with said sealing element (71).

According to a further non-limiting aspect, the bottom wall (72) and the sealing element (71) upperly delimit the tank (6).

According to a further non-limiting aspect, the sealing element (71) comprises an annular wall radially arranged in an outer position of the bottom wall (72) or radially protruding from said bottom wall (72).

According to a further non-limiting aspect, said annular wall comprises a radially outer portion exerting a friction, in particular a sliding friction, during the movement of the second body (7) between the first and the second position.

According to a further non-limiting aspect, the stopper (1) comprises a peelable element (80) arranged in substantial correspondence of the first portion (4) of the first body (4, 5).

According to a further non-limiting aspect, the peelable element (80) is configured and specifically destined to act as a barrier from the contact or against an extraction of said substance (2) when leaking from the tank (6) through the at least one discharge hole (9).

According to a further non-limiting aspect, the peelable element (80) comprises a sheet of flexible and/or bendable material, optionally comprising at least one between a layer of plastic material and a layer of metallic material, preferably aluminium.

According to a further non-limiting aspect, the peelable element (80) is in use united to said first portion (4) of the first body (4, 5) through at least one between a gluing process and/or a welding process, said welding being preferably an ultrasonic welding.

According to a further non-limiting aspect, the second portion (5) of the first body (4, 5) comprises an engagement portion (59) with the first portion (4) of the first body (4, 5).

According to a further non-limiting aspect, the engagement portion (59) is configured to maintain movably constrained one end of the second portion (5), preferably a lower end of the second portion (5), with an end of the first portion (4), preferably an upper end of the first portion (4).

According to a further non-limiting aspect, the engagement portion (59) is configured and specifically destined to determine and maintain a centering of the second portion (5) on the first portion (4), preferably being configured and specifically destined to determine and maintain a centering of the movement of the second portion (5) with respect to the first portion (4) between said first relative position and said second relative position; said centering optionally occurring on the main development axis (X) of the stopper (1).

According to a further non-limiting aspect, the engagement portion (59) comprises at least one annular profile, extending seamlessly from the lower side of the dividing wall (5p) of the second portion (5).

According to a further non-limiting aspect, the first portion (4) comprises an engagement area (48) for the engagement portion (59).

According to a further non-limiting aspect, the engagement area (48) is realized in substantial correspondence of a recess (43) opening in substantial correspondence of the dividing wall (4p) of the first portion (4).

According to a further non-limiting aspect, the engagement area (48) comprises an undercut suitable for housing at least part of a protrusion of the engagement portion (59).

According to a further non-limiting aspect, the protrusion of the engagement portion (59) extends in a direction substantially oblique with respect to the direction identified by the main development axis (X) of the stopper (1).

According to a further non-limiting aspect, said recess (8) opens on a cavity (8c) configured to allow the removable engagement of an end portion of a rod (100) by introduction by contrast.

According to a further non-limiting aspect, said stopper is configured to allow the introduction by contrast only when in open configuration, in particular in substantial proximity or correspondence of the configuration of minimum axial extension.

According to a further non-limiting aspect, the cavity (8c) has a plurality of retaining ribs (8n) of the rod (100).

According to a further non-limiting aspect, the ribs (8n) radially protrude towards the center of the cavity (8c) itself and/or are substantially aligned along an axis substantially coinciding with the longitudinal axis of the stopper (1).

According to a further non-limiting aspect, said ribs (8n) are configured to exert a contrasting force at the insertion and extraction of part of the rod (100) with respect to the cavity (8c) and to help the transfer of at least part of the substance (2) from the tank (6) towards the outside of the stopper (1).

According to a further non-limiting aspect, the stopper (1) comprises at least one positional reference element (94), configured and specifically destined to identify a predefined spatial orientation of the stopper (1).

According to a further non-limiting aspect, said at least one positional reference element (94) defines at least one predefined accessible outer portion of the stopper (1), in particular a first accessible outer portion of at least one between the first body (4, 5) and the second body (7) non-axisymmetric with a second accessible outer portion of said at least one between said first body (4, 5) and said second body (4, 5).

According to a further non-limiting aspect, said at least one positional reference element (94) is configured and specifically destined to identify an orientation of the stopper (1) with respect to the rotation axis (X).

According to a further non-limiting aspect, said at least one positional reference element (94) is arranged in correspondence of the first portion (4) of the first body (4, 5) and/or is arranged in correspondence of the second portion (5) of the first body (4, 5) and allows to identify a reference position of the first portion (4) of the first body (4, 5) and/or of the second portion (5) of the first body (4, 5) with respect to the rotation axis (X).

According to a further non-limiting aspect, said at least one positional reference element (94) comprises a notch.

According to a further non-limiting aspect, said positional reference element (94) is realized on an outer surface of the first portion (4) of the first body (4, 5) and/or on an outer surface of the second portion (5) of the first body (4, 5).

According to a further non-limiting aspect, said predefined accessible outer portion of the stopper (1) is part of the outer surface of the first portion (4) of the first body (4, 5) and/or is part of the outer surface of the second portion (5) of the first body (4, 5).

According to a further non-limiting aspect, said at least one positional reference element (94) comprises at least one among a notch, a protrusion, a graphic or alphanumeric code.

According to a further non-limiting aspect, said graphic code or said alphanumeric code is substantially co-planar with said predefined accessible outer portion of the stopper (1).

### Kit

According to the present disclosure it is furthermore described a kit comprising:

a stopper (1) according to one or more of the preceding aspects,
and at least one among:
a device (100, 101) for collecting samples, preferably a device for collecting samples of biological and/or microbiological material, comprising a rod (100) and a swab (101), joined with, at least partially superimposed on, realized on, said rod (100), and preferably configured and specifically destined to allow the collection and/or the storage of a sample of biological and/or microbiological material;
a container (3) for samples, preferably a container (3) for samples of biological and/or microbiological material, the container being configured and specifically destined to be removably coupled to said stopper (1) in correspondence of an opening, and being specifically destined to house said device (100, 101) for collecting samples;
an adapter, configured and specifically destined to be removably coupled to the stopper (1) in correspondence of the first body (4, 5), optionally in correspondence of the first portion (4) of the first body (4, 5); said adapter being configured and specifically destined to removably couple the stopper (1) to an auxiliary device, preferably to at least one between an auxiliary container for samples, a duct for the release of the substance (2), a cartridge.

### Method of Handling

According to the present disclosure it is furthermore described a method of handling of a stopper (1) according to one or more of the aspects herein described.

According to a further aspect, the method comprises:

a first step of engagement of the first portion (4) of the first body (4, 5),
a second step of engagement of the second portion (5) of the first body (4, 5),
a first step of movement, comprising a movement of the first portion (4) with respect to the second portion (5) between said first relative position and said second relative position, the step of movement occurring after the first and the second step of engagement;
a step of engagement of the second body (7);
a second step of movement, subsequent to the first step of movement, the second step of movement comprising a movement of the second body (7) with respect to the first body (4, 5) between said first position and said second position;
the second step of movement determining a leakage at least partial of the substance (2) from the tank (6) through said at least one discharge hole (9);
the method of handling being optionally a method of handling at least partially automated and being carried out by a machine for handling of stoppers (1) for releasing a substance (2) into a container (3) for samples, preferably a container (3) for samples of biological and/or microbiological material.

According to a further non-limiting aspect, the first step of movement comprises a rotation of the first portion (4) with respect to the second portion (5) between said first relative position and said second relative position and/or the second step of movement comprises a linear translation of the second body (7) with respect to the first body, preferably along a direction substantially parallel to a main development axis (X) of the stopper (1).

According to a further non-limiting aspect, said rotation occurs in a predefined sense of rotation starting from a first end stroke position assumed by the first portion (4) of the first body (4, 5) with respect to the second portion (5) of the first body (4, 5).

According to a further non-limiting aspect, said rotation ends in correspondence of a second end stroke position assumed by the first portion (4) of the first body (4, 5) with respect to the second portion (5) of the first body (4, 5).

According to a further non-limiting aspect, said rotation determines a movement of a closing element (10) of at least one discharge hole (9) of the stopper (1) from a first position wherein said closing element (10) closes said at least one discharge hole (9) determining a closed configuration of said tank (6) to a second position wherein said closing element (10) opens said at least one discharge hole (9) determining an open configuration of said tank (6).

According to a further non-limiting aspect, the linear translation of the second body (7) with respect to said first body (4, 5) determines a progressive reduction of the volume of the tank (6) and/or a progressive leakage of the substance contained in the tank (6).

According to a further non-limiting aspect, the linear translation of the second body (7) with respect to said first body (4, 5) ends when at least part of the second body (7) enters in contact with an end stroke element positioned in correspondence of the first body (4, 5), said end stroke element being optionally a bottom wall of the tank (6).

According to a further non-limiting aspect, the method comprises a removal of a peelable element (80) arranged in substantial correspondence of the first portion (4) of the first body (4, 5).

According to a further non-limiting aspect, the removal of the peelable element (80) occurs before the second step of movement.

According to a further non-limiting aspect, the removal of the peelable element (80) occurs before the first step of movement.

### Machine

According to the present disclosure it is furthermore described a machine, configured and specifically destined to engage removably the stopper (1) according to one or more of the aspects herein described.

According to a further non-limiting aspect, the machine comprises at least one handler configured to determine a relative movement of the first portion (4) of the first body (4, 5) with respect to the second portion (5) of the first body (4, 5) at least between said first relative position and said second relative position and/or to determine a relative movement between said second body (7) and said first body (4, 5) between said first position and said second position.

According to a further non-limiting aspect, said handler is configured to temporarily retain said first portion (4) of the first body (4, 5) in a predefined fixed position and to move the second portion (5) of the first body (4, 5).

According to a further non-limiting aspect, the machine is configured and specifically destined to determine the execution of the method according to one or more of the above-described aspects.

According to a further non-limiting aspect, the machine is specifically configured, optionally automatically, to:
engage the first portion (4) of the first body (4, 5) through said at least one handler and engage the second portion (5) of the first body (4, 5) through said at least one handler;
subsequently, move, optionally rotate, the first portion (4) of the first body (4, 5) with respect to the second portion (5) of the first body (4, 5) through said at least one handler to open said tank (6), optionally to free said at least one discharge hole (9) from said at least one closing element (10);
engage, through said at least one handler, the second body (7);
then, axially push the second body (7), so as to move it from the first position relatively assumed with respect to the first body (4, 5), in particular with respect to the second portion (5) of the first body (4, 5) towards the second portion relatively assumed with respect to the first body (4, 5), in particular with respect to the second portion (5) of the first body (4, 5), so as to determine a progressive reduction of the volume of the tank (6) and to force the leakage of the substance (2) from the at least one discharge hole (9).

### FIGURES

The object of the present disclosure will be now described in preferred and non-limiting embodiments described with the support of the attached figures, a short description thereof is hereinafter provided.

FIG. 1 shows a perspective view in exploded view of the stopper object of the present disclosure.

FIG. 2 shows a sectional perspective view of the stopper object of the present disclosure, in a configuration wherein its tank is closed.

FIG. 3 shows a sectional perspective view of the stopper object of the present disclosure, in a configuration wherein its tank is open; in FIG. 3, the tank, of variable volume, presents a first volume.

FIG. 4 shows a sectional perspective view of the stopper object of the present disclosure, in a configuration wherein its tank is open; in FIG. 4, the tank, of variable volume, presents a second volume smaller with respect to the first volume.

FIG. 5 shows a perspective view from above of a first portion of a first body of the stopper object of the present disclosure.

FIG. 6 shows a plan view, from the bottom, of the first portion of the first body of the stopper object of the present disclosure.

FIG. 7 shows a plan view from above of a second portion of the first body of the stopper object of the present disclosure.

FIG. 8 shows a plan view from the bottom of the second portion of the first body of the stopper object of the present disclosure.

FIG. 9 shows a section view along the line Y-Y of FIG. 7.

FIG. 10 shows a section view of the second portion of the first body, in a first relative configuration of rotation with respect to a first portion of the first body.

FIG. 11 shows a section view of the second portion of the first body, in a second relative configuration of rotation with respect to a second portion of the first body.

### DETAILED DESCRIPTION

The present disclosure concerns a stopper 1 for releasing a substance 2 into a container 3. Preferably, the stopper 1 is destined to be coupled with a container 3 for biological and/or microbiological material and to release the substance 2 into said container.

In an embodiment, the stopper 1 is specifically conceived to be used by a user who can be a health assistant (for example a doctor or a nurse) or a patient. The stopper 1 object of the present disclosure is in particular conceived to be used in combination with containers 3 for self-testing.

The stopper 1 object of the present disclosure comprises a first body 4, 5, which, in its entirety, is destined to be removably engaged on the container 3. The stopper 1 also comprises a second body 7 coupled and movable with respect to the first body 4, 5.

The stopper 1 is provided with a tank 6 configured to contain the substance 2; the tank 6 is defined between the first body 4, 5 and the second body 7. In an embodiment, the tank 6 is realized by the first body 4, 5 and by the second body 7 without additional elements introduced. As it will be made clear in the following description, if the second body 7 is forced into disconnection or separation from the first body 4, 5, the tank 6 results open. In this case, the tank 6 can be conveniently emptied or filled, depending on the need.

In a non-limiting embodiment, the container 3 can be for example and in a non-limiting extent a test tube, to contain and/or preserve a biological sample, possibly to be later subjected to one or more analyses. In particular, the coupling between the stopper object of the present disclosure and the container 3 occurs in correspondence of an opening of the container itself, which in an embodiment herein described—and shown in the attached figures—is also the only opening of the container itself.

The stopper 1 object of the present disclosure can be referred to as “releasing” since configured to contain a substance 2 within a tank thereof, and release in a predetermined way, the substance 2 from its tank towards the container 3. The substance 2 contained in the tank 6 is a substance which can be both fluid, in particular liquid, and in solid form, for example in granular and/or powder form. The above-described substance 2 can be or comprise, in addition to solid and liquid, also a gelatinous substance, and/or can comprise Newtonian and/or non-Newtonian fluids, and/or can be or comprise gases or vapours. The stopper 1 object of the present disclosure is therefore capable of containing any form of material without limitation. In particular, the stopper object of the present disclosure may also be capable and/or destined to contain a substance 2 emitting ionizing radiation.

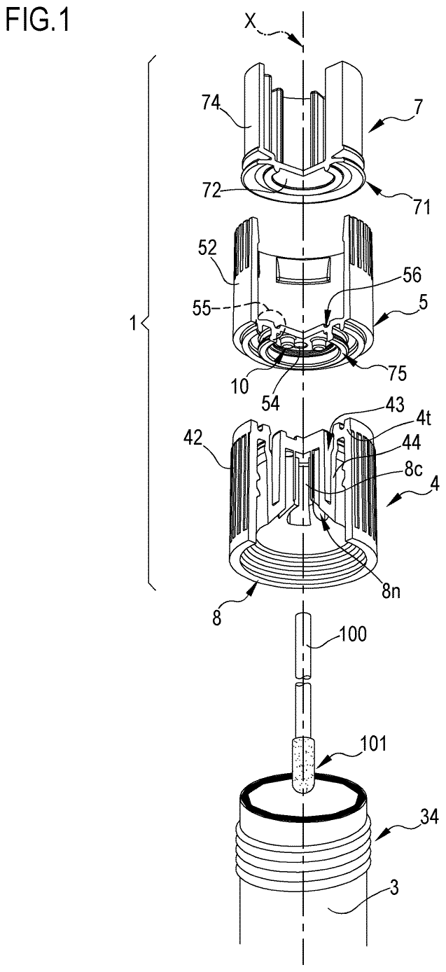

In a preferred but non-limiting embodiment, the container 3 is substantially a test tube for the collection and/or storage and/or analysis of biological samples, preferably realized in plastic material, for example but in a non-limiting extent PP or PET or other plastic material, preferably conceived in order to not to release substances towards the biological sample when contained therein. In particular it can be a test tube of plastic material, optionally transparent to visible optical radiation, with a size for example but in a non-limiting extent of 12 mm×80 mm. This shape is not to be intended as limiting. In the configuration shown in FIG. 1, the container 3 presents a substantially cylindrical body open in correspondence of a single end (upper end) in correspondence of which there is the threading.

In the preferred embodiment, the stopper 1, and in particular the first body 4, 5 and the second body 7 have substantially a shape substantially of a solid of revolution, in particular identifying a substantially circular outer profile. An axis X is centered on the stopper 1 and is a center axis of the first body 4, 5 and of the second body 7. As it will be better explained in the following portion of the description, the axis X becomes the translation axis of the second body 7 with respect to the first body 4, 5.

The tank 6 comprises a bottom wall which acts as an end stroke element for the second body 7 during its linear translation with respect to the first body 4, 5, through which the volume of the tank 6 is progressively decreased starting from a predefined volume, preferably a maximum volume of the tank 6, and/or through which the substance contained in the tank is progressively pushed out thereof.

The first body 4, 5 comprises a first portion 4 and a second portion 5. The axis X is the axis on which it occurs a movement, in particular a rotation, of the first portion 4 with respect to the second portion 5.

### The First Portion 4 and the Second Portion 5

The first portion 4 of the first body 4, 5 is configured to be directly coupled on the container 3 and comprises a coupling element 41 configured to allow the removable fastening of the stopper 1 in correspondence of an opening of the container 3. When the coupling element 41 is fixed to the opening of the container 3 a first mounting configuration of the stopper 1 on the container is outlined.

The second portion 5 of the first body 4, 5 is located between the first portion 4 of the first body 4, 5 and the second body 7. In particular, the second portion is movably connected with the first portion 4 and realizes at least one part of a bottom wall and/or of a lateral wall of the tank 6.

In an embodiment, whatever the configuration of the stopper may be, at least part of the second portion 5 of the first body 4, 5 results in an intermediate position between the first portion 4 of the first body 4, 5 and the second body 7.

In an embodiment, the coupling element 41 can be a threading, which preferably but in a non-limiting extent is arranged on an inner side of a coupling wall 42, or outer lateral wall, of the first portion 4 of the first body 4, 5. This should not be intended in a limiting way, since in a non-limiting and alternative embodiment, the coupling element 41 can be arranged on an outer side of the coupling wall 42 of the first portion 4 of the first body 4, 5.

It is noted that the container 3 comprises a threading 34 in correspondence of which the stopper 1 removably engages. The threading 34 is arranged in substantial correspondence of the opening of the container 3. In the embodiment wherein the coupling element 41 is arranged on the inner side of the coupling wall 42, the threading 34 is arranged on the outer side of the portion of container 3 substantially corresponding to the opening. In the embodiment wherein the coupling element 41 is arranged on the outer side of the coupling wall 42, the threading 34 is arranged on the inner side of the portion of container 3 substantially corresponding to the opening. In other words, this means that the container 3 can be a container with inner threading or, alternatively, a container with outer threading.

The first portion 4 of the first body 4, 5 comprises a dividing wall 4p which is positioned in a substantially transverse direction with respect to the direction along which it extends the coupling wall 42, and it is therefore arranged substantially on a plane orthogonal with respect to the axis X. The dividing wall 4p is substantially a central wall of the first portion 4, and is centered on the axis X.

In an embodiment the dividing wall 4p presents, along a plane substantially orthogonal with respect to the axis X, an almost circular shape and comprises a first and a second discharge hole 9. The first and the second discharge hole 9 are each arranged at a same distance from the axis X. The presence of two discharge holes 9 should not be intended in a limiting way, since in an alternative embodiment, the discharge hole 9 can be one; in this case, preferably, the discharge hole 9 can be arranged in substantial alignment to the axis X.

Between the dividing wall 4p and the coupling wall 42 extends a substantially annular recess 43 defined in correspondence of an intermediate wall 44, which when observed on a plane substantially including the axis X, identifies a substantially “U” shape, identifying an annular recess 43. The recess 43 opens in substantial correspondence of the dividing wall 4p.

The dividing wall 4p comprises an upper side and a lower side. In correspondence of the upper side of the dividing wall 4p there are two substantially opposed recesses 11. Each recess 11 comprises a first end and a second end. The first end of each recess opens in substantial correspondence of an its own discharge hole 9. The second end of each recess ends in a portion of the dividing wall 4p away from the discharge hole 9. In correspondence of the second end of each recess can be present a small blind hole 11s, of profile substantially equal to, or slightly greater than, the one of the discharge hole 9. In the attached figures, the closing element 10 has a circular profile; therefore, also the blind hole 11s or blind drilling will preferably have a circular profile. The blind hole 11s or blind drilling is arranged in correspondence of a bottom, or main, wall of the recess 11. This wall extends substantially on a plane orthogonal with respect to the axis X.

In an alternative embodiment, at least one recess 11, or the two above-described recesses 11, could be arranged on a different wall with respect to the dividing wall 4p; for this reason, the arrangement of the recesses 11 on the dividing wall 4p should not be intended in a limiting way. In particular the configuration of the closing element 10, of the discharge holes and of the recesses 11 could be inverted with respect to the embodiment represented in the attached figures, and for this reason the recess 11 could be located on the lower side of the dividing wall 5p of the second portion 5 of the first body 4, 5.

The substantially elongated shape of the two recesses 11 identifies a first end stroke position and a second end stroke position for the movement, in particular for the rotation, realizable between the first portion 4 and the second portion 5 of the first body 4, 5. The first end stroke position is determined when the closing element 10 of the discharge hole 9 (better described below) is in correspondence of the first end of the respective recess. The second end stroke position is determined when the closing element is in correspondence of the second end of the respective recess.

In a non-limiting embodiment, the first portion 4 and the second portion 5 of the first body 4, 5 are configured to rotate reciprocally by a rotation angle predefined by the first end stroke position and by the second end stroke position. Preferably, although in a non-limiting extent, this angle is lower than 360°, preferably is lower than 180° and more preferably is substantially equal to a ¼ turn (90°).

The two recesses 11 present a shape outlining a circumference arc along a plane orthogonal with respect to the axis X, therefore along a plane orthogonal to the rotation axis relatively existing between the first portion 4 and the second portion 5 of the first body 4, 5. The shape of the two recesses 11 (or of the single recess, where present) is conceived in order to allow to guide the first portion 4 of the first body 4, 5 in rotation with respect to the second portion 5 of the first body 4, 5 with a correct positional relationship.

Should the specific embodiment of the first portion 4 of the first body 4, 5 present a single discharge hole 9, there will be a single recess 11 on the dividing wall 4p. This single recess 11 will have, preferably, the shape of a circumference arc too.

The Applicant in particular observes that in correspondence of the first end of the recess 11 (where they are present in a number equal to two, in correspondence of the first ends of the recess 11) there is a connection portion 11r which presents a sliding surface for the closing element which is curved and without angular points. The connection portion 11r is for example visible in the FIG. 5.

As it is better explained in the following portion of description, since the opening of the tank 6 occurs by freeing the discharge hole 9 by relative movement, in particular by relative rotation, between the first portion 4 and the second portion 5 of the first body 4, 5, the connection portion 11r advantageously allows to facilitate the leakage of a closing element which is introduced within the discharge hole 9 in case the tank is in closed configuration.

The connection portion 11r is configured and specifically destined to determine a variation of height assumed by the closing element 10 of the discharge hole 9 (better described below), in particular along the main development axis X of the stopper, during the movement of the first portion 4 of the first body 4, 5 with respect to said second portion 5 of the first body 4, 5 between said first relative position and said second relative position. This is made possible thanks to the deformability and/or flexibility of the dividing wall 5p on which the closing element 10 is arranged.

Preferably, and independently of the presence or otherwise of the connection portion 11r the stopper 1 object of the present disclosure is configured to allow the extraction of the at least one closing element 10 from the respective at least one discharge hole 9 through a snap; in other terms, the movement of the closing element 10 from the first end stroke position occurs through said snap. For the purposes of the present disclosure, said snap turns into a greater release force with respect to a force otherwise required to complete at least part of the rotation of the first portion 4 of the first body 4, 5 with respect to the second portion of the first body 4, 5. This advantageously allows to have a greater security in the effective will to operate in rotation the first portion 4 of the first body 4, 5 with respect to the second portion 5 of the first body 4, 5.

This snap is determined by a shoulder, which preferably assumes a substantially squared profile, between the at least one discharge hole 9 and the connection portion 11r.

The blind hole 11s allows to maintain the closing element 10 in correspondence of the second end of the recess 11, reducing the risk of an inadvertent relative movement between the first and the second portion of the first body 4, 5 in opposed direction with respect to before, which could again determine an access of the closing element within the discharge hole 9. In particular, the blind hole 11s acts as an element configured and specifically destined to prevent a counterrotation of the second portion of the first body 4, 5 with respect to the first portion of the first body 4, 5 or to reduce the risk of such counterrotation, mainly if unwanted, as a result of the movement of the closing elements 10 from the first end stroke position to the second end stroke position. The leakage of the closing element 10 from the blind hole 11s requires a counterrotation force of the first portion 4 of the first body 4, 5 with respect to the second portion 5 of the first body 4, 5 much greater than the one that would otherwise be required to carry out the remaining part of rotation with the closing element 10 positioned in the recess 11 but out of the blind hole 11s. For this reason, the blind hole 11s may also be referred to as safety or engagement element, or anti-counterrotation element. Having defined as direct rotation the rotation which allows the displacement of closing elements 10 from the first end stroke position to the second end stroke position, the undercut 11 then acts as a blocking element of the reverse rotation, which is consequently defined as that rotation which would allow the displacement of the closing elements 10 from the second end stroke position towards the first end stroke position.

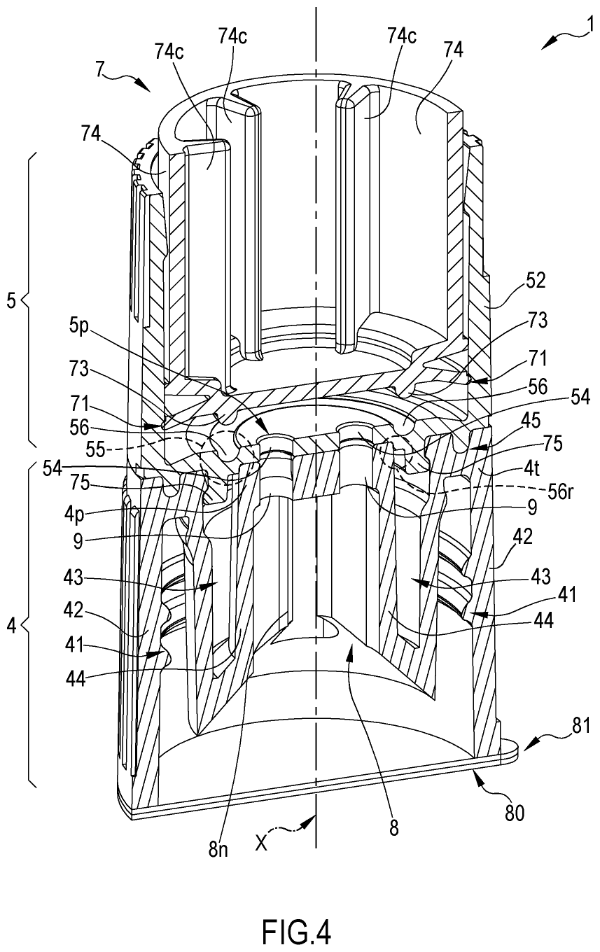

With reference to FIG. 4, or to FIG. 5, the direct rotation—keeping fixed the position of the first portion 4 of the first body 4, 5—is a clockwise rotation of the second portion 5 of the first body 4, 5 with respect to the first portion 4 of the first body 4, 5.

By summarizing, both the previously disclosed snap, and the presence of the blind hole 11s as a blocking element of the reverse rotation, allow to reduce the risk of unintentional opening of the tank 6 and subsequently the new closing thereof.

The first portion 4 of the first body 4, 5 also comprises a head wall 4t which is joined in a radially outer position with an upper end portion of the coupling wall 41 or outer lateral wall.

In correspondence of the upper side of the head wall 4t it can be preferably present a guiding element 45, in the form of a substantially annular recess. The guiding element 45 is configured to allow to guide a rotation relative between the first portion 4 and the second portion 5 of the first body 4, 5 around an axis substantially coinciding with the axis X. The guiding element 45 cooperates in use, during the relative rotation between the first portion 4 and the second portion 5, with the annular recess 43 and with the at least one recess 11 present in correspondence of the dividing wall 4p.

If the threading is arranged on the inner side of the coupling wall 42, on the outer side of the coupling wall 42, opposed with respect to the inner side, can be present elements—knurling, even alternating, recesses or raised portions—destined to promote the grip of the user's fingers. Preferably such elements extend seamlessly along the entire perimeter of the outer face of the coupling wall 42.

In correspondence of the lower part of the first portion 4 of the first body 4, 5 is present a hollow 8 in the shape of substantially a bell wherein it is obtained a recess 8c or cavity. This cavity 8c is destined to realize a housing suitable for containing in use an end portion of a rod 100, in particular retaining it by introduction by contrast; to this purpose, the inner surface of the cavity 8c is provided with a plurality of retaining ribs 8n, preferably but in a non-limiting extent arranged each one along a direction parallel to the direction identified by the axis X and protruding towards the center of the cavity 8c itself, identified precisely by the axis X.

Suitable spacers are positioned within the cavity 8c to allow to axially separate the discharge holes 9 from the upper end portion of the rod 100 when it is fully introduced within the recess 8c.

Observed from below, in fact, the first portion 4 of the first body 4, 5 identifies a substantially helix-shaped recess or cavity 8c. At least two, preferably four, retaining walls which realize a plurality of retaining ribs 8n protrude radially inwards, and therefore towards the axis X, in order to identify a contact point with an end portion of the rod 100 in correspondence of which is developed a sliding friction having a force sufficient to be greater with respect to the entire weight of the device for collecting samples, preferably for collecting biological and/or microbiological material (including the weight of the sample itself), so that it can be firmly retained without risk of separation in any operating configuration.

When two, or four, retaining walls which realize the plurality of retaining ribs are opposed to each other and in particular radially opposed with respect to a development axis of the first portion 4 of the first body 4, 5.

In the embodiment shown in the attached figures, the retaining ribs 8n are two by two opposed. This configuration should not be intended as limiting, since the Applicant has conceived a further embodiment wherein the retaining ribs 8n are three, oriented by 120° on three directions orthogonal with respect to the axis X. The number of ribs is clearly not limiting.

If observed along a plane orthogonal with respect to the axis X, the radially inner portion of the retaining ribs 8n presents a blade profile (pointed), and the four more radially inner portions of the retaining ribs 8n identify circumferential arcs of an ideal circumference having a diameter slightly smaller with respect to the diameter of the rod 100. A radially outer portion of the retaining ribs 8n, opposed with respect to the radially inner portion, is joined seamlessly with the lateral wall of the cavity 8c.

In use, at the insertion of the rod 100, the most radially inner portions of the retaining ribs 8n elastically deform, retaining the rod. In use, the upper end portion of the rod 100 is introduced within the cavity 8c and is retained thereby. Consequently, when the operator extracts or otherwise separates the stopper 1 from the container 3, the rod 100 is retained together with the stopper itself.

The stopper 1 acts then as a handle for the rod 100; this technical effect advantageously reduces the risk of contamination between rod and operator, and conveniently prevents that the latter can in turn be contaminated with portions of the collected sample and/or with residues of the substance 2.

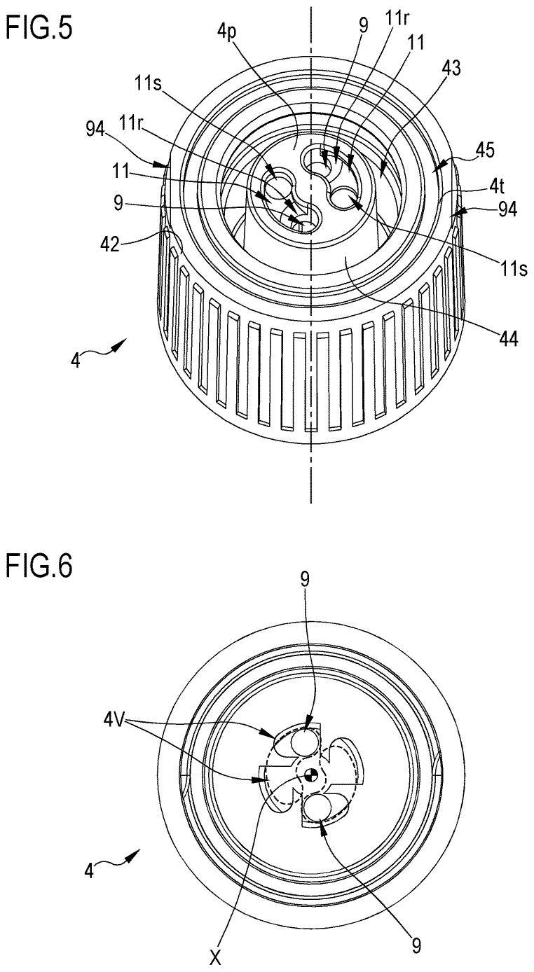

As schematically shown in FIG. 6, channels 4v are present between the retaining ribs 8n, and are realized, in the preferred embodiment, by the “blades” of the helix. At least two of said channels 4v, and preferably all the four channels 4v, directly communicate with the two discharge holes 9 (or with the single discharge hole 9, in the relative embodiment).

The channels 4v are positioned in a radially outer portion with respect to the portion of the cavity 8c which in use houses the rod 100, and in the embodiment shown in the attached figures are realized between two contiguous retaining ribs 8n.

The Applicant has, however, realized further embodiments, not shown in the attached figures, wherein the channels 4v do not communicate with the cavity 8c, but are external to it or otherwise separated from the cavity 8c. In these embodiments, the channels 4v can be arranged parallel to the main development direction of the cavity 8c.

The substance coming out of the discharge holes 9 (or from the single discharge hole 9, in the relative embodiment), transits along the channels, sliding along a direction substantially parallel to the axis X, and is pushed into an outer zone with respect to the lateral surface of the rod 100.

It is noted in FIG. 4 and/or in FIG. 6, that the discharge holes 9 are at a radial distance from the axis X greater with respect to the radius of the ideal circumference identified by the radially inner portions of the retaining ribs 8n. This solution is not to be intended as limiting, since in an alternative embodiment, the discharge holes 9 could be at a radial distance from the axis X smaller with respect to the radius of the ideal circumference identified by the radially inner portions of the retaining ribs 8n.

Preferably but in a non-limiting extent, the first portion 4 comprises an engagement area 48 destined to house an engagement portion 59 of the second portion 5. This engagement portion 59 is destined to promote the centering of the movement, in particular of the rotation, between the first and the second portion. The engagement area 48 is preferably realized in substantial correspondence of the recess 43, which opens in substantial correspondence of the dividing wall 4p and which is defined through the intermediate wall.

Preferably, the engagement area 48 comprises an undercut suitable for housing a protrusion of the engagement portion of the second portion 5.

The second portion 5 of the first body 4, 5 presents a lateral wall 52 that in a preferred embodiment assumes a substantially circular transversal section. The lateral wall 52 can identify a substantially cylindrical shape or a truncated coned shape. The lateral wall 52 comprises an outer side and inner side. On the outer side of the lateral wall 52, similar to what occurs for the coupling wall 42, can be present elements—knurling, even alternating, recesses or raised portions—destined to promote the grip of the user's fingers. Preferably such elements extend seamlessly along the entire perimeter of the outer face of the lateral wall 52.

The second portion 5 of the first body 4, 5 also comprises a bottom wall 5p or dividing wall. Preferably at least part of the bottom wall 5p, or dividing wall, is arranged on a plane substantially orthogonal with respect to the axis X; similarly to the dividing wall of the first portion 4 of the first body 4, 5, also the dividing wall 5p of the second portion 5 is substantially centered on the axis X. The purpose of the dividing wall 5p is to realize a bottom wall of the tank 6 and to therefore subdivide in a substantial way the tank 6 from the first portion 4 of the first body 4, 5.

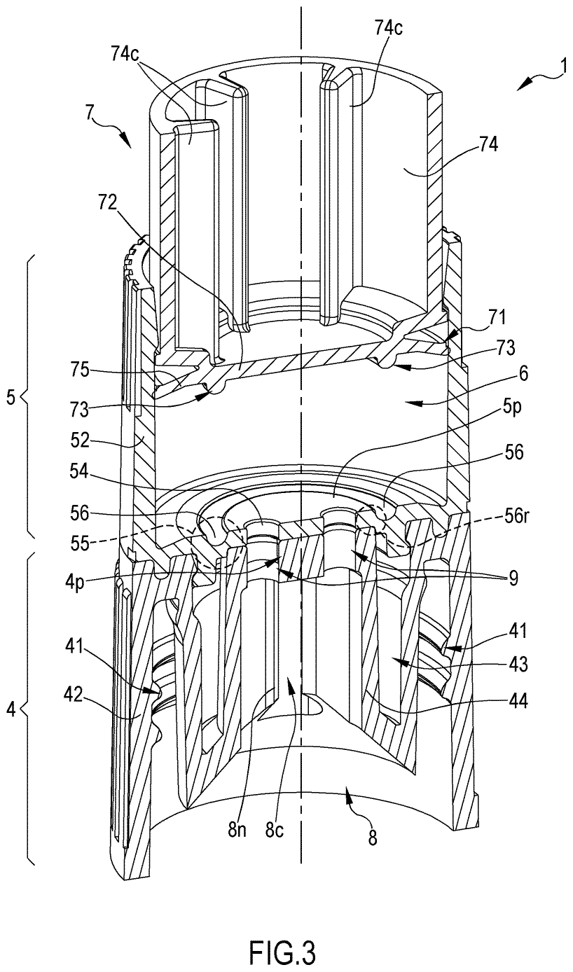

The inner side of the lateral wall 52 is substantially smooth and presents a preferably substantially circular transverse section. This allows an easy relative sliding of the second body 7 into the lateral wall 52, being able therefore to determine a variation of volume of the tank 6. In this regard, see the difference between FIG. 3 and FIG. 4, where in the latter the second body 7 is introduced more into the lateral wall 52.

The dividing wall 5p comprises at least one through hole 54. This through hole 54 is configured to allow the extraction of the substance 2 from the tank 6 by putting in communication the tank 6 with the at least one discharge hole 9. In the embodiment shown in the attached figures, there are two through holes 54, the radial distance thereof with respect to the axis X is equal to the radial distance assumed by the drain holes 9 present in the second portion 5 of the first body 4, 5.

The direction along which the through holes 54 develop is preferably parallel to the axis X.

The dividing wall 5p comprises an inner or upper side, that is the one that delimits the tank 6, and an outer or lower side, opposed with respect to the inner or upper side. The outer or lower side is the one that in use is in substantial proximity or contact with the dividing wall 4p of the first portion 4 of the first body 4, 5.

The dividing wall 5p can be substantially rigid or flexible, in particular the bending occurring along the axis X. The dividing wall 5p develops overall on a plane substantially orthogonal with respect to the axis X and by effect of this bending the plane moves along the axis X itself.

Preferably, although in a non-limiting extent, the dividing wall 5p presents a weakening portion 55 in the form of a recess substantially annular wherein the thickness of the dividing wall 5p can be reduced or wherein the particular conformation of this dividing wall locally, for example through a hump with convexity directed downwards, is such as to promote the above-described bending.

As for example shown in FIG. 9, in correspondence of the weakening portion 55 is realized an annular recess 56 with corresponding raised portion 56r which protrudes in convexity on the lower side or outer side of the dividing wall 5p.

The raised portion 56r in use inserts in the annular recess realized by the guiding element 45 on the head wall 4t.

The dividing wall 5p is conceived in particular in order to have an elastic deformability such as to determine a local alteration of the height thereof along the direction identified by the axis X. This elastic deformability is in particular given by the weakening portion 55.

On the lower or outer side of the dividing wall 5p are present a first and a second raised closing element 10, the shape thereof follows that of the draining holes 9. The first and the second closing element, preferably of circular shape, are realized integrally on the dividing wall 5p, preferably, but in a non-limiting extent, through an injection moulding process.

The deformability of the dividing wall 5p allows the variation of the height of the closing element 10 along the axis X and allows the leakage of the closing element 10 from the respective discharge hole 9 downwards of the rotation of the first portion 4 of the first body 4, 5 with respect to the second portion 5 of the first body 4, 5.

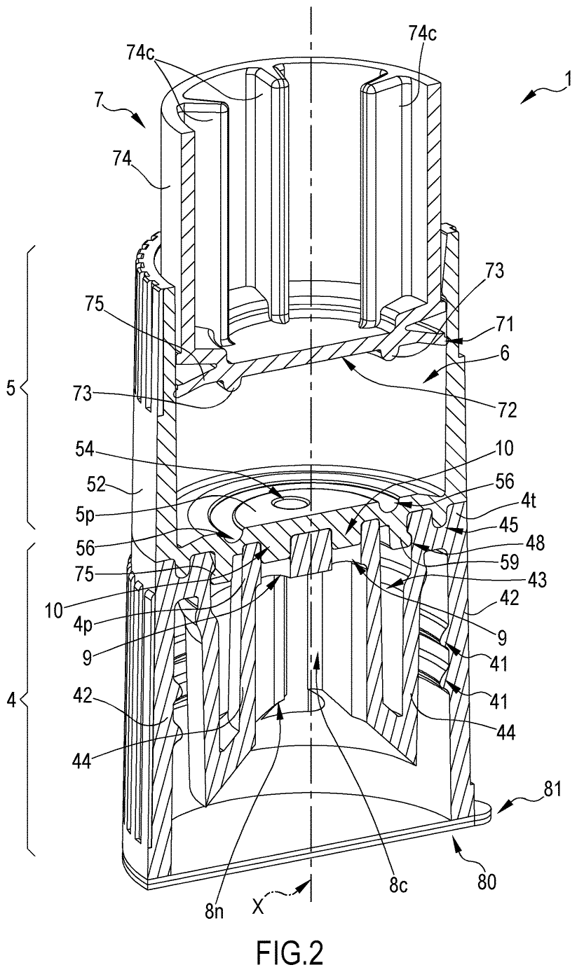

The first and the second closing element 10 protrude downwards, along a direction identified by the main development axis X of the stopper 1. In particular, this first and second closing element 10 protrude substantially orthogonally, at least locally, with respect to the dividing wall 5p. In the embodiment represented in the attached figures, the first and the second closing element 10 are substantially of cylindrical shape.

Where there is only one single discharge hole 9, there will be only one first closing element. The through hole 54 is configured to couple with the respective discharge hole 9 alternatively to the closing element 10 as a result of the relative movement between the first portion 4 and the second portion 5 of the first body 4, 5. The coupling between the through hole 54 and the respective discharge hole 9 is an axial coupling. As it will be better described below, and as it is visible for example in FIGS. 3 and 4, when the through hole 54 and the respective discharge hole 9 are coupled, they are aligned along an axis substantially parallel to the axis X. FIG. 2 shows instead a configuration wherein the through hole 54 is not aligned with a respective discharge hole 9.

The first and the second closing element 10 are specifically arranged and configured to allow the closing of the draining holes 9. In the embodiments shown in the attached figures, the first and the second closing element 10 have a shape having a transverse section which is substantially circular.

In order to allow the closing of the draining holes 9 as a result of the relative movement between the first portion 4 and the second portion 5 of the first body 4, 5, in a preferred but non-limiting embodiment, the radial distance from the axis X of each closing element 10 is equal to the radial distance from the axis X assumed by the draining holes 9.

When the first portion 4 and the second portion 5 of the first body 4, 5 are assembled to each other, the closing elements 10 lie within the recesses 11.

Preferably, but in a non-limiting extent, the second portion 5 of the first body 4, 5 is realized in plastic material and is realized through known moulding technique, not described in detail herein (for example, injection moulding, co-moulding). Being the second portion 5 in contact with the substance 2, the plastic material with which it is realized is attentively selected in order to be compatible, and not to react, with the substance 2. In an embodiment, the second portion 5 of the first body 4, 5 is realized as a piece.

An upper portion, or end portion, of the inner side of the lateral wall 52, is provided with a shoulder 5k radially protruding inwards; this shoulder 5k acts as contrasting element against the complete extraction of the second body 7 from the second portion 5 of the first body 4, 5.

Preferably, in correspondence of the lower side of the dividing wall 5p of the first portion 5 there is an engagement portion 59, that is suitable for engaging the previously described engagement area 48. Preferably, this engagement portion 59 protrudes from the lower side of the dividing wall 5p, but this protruding configuration is not to be intended as limiting since the engagement portion 59 could protrude towards the inside of the dividing wall 5p.

Whether this engagement portion 59 protrudes outwards, or this engagement portion 59 protrudes inwards, preferably it presents an annular profile that allows to realize the relative rotation between the first portion 4 and the second portion 5 of the first body 4, 5. From the engagement portion 59 departs a protrusion which engages the undercut of the engagement area 48. The protrusion extends at least partially in an oblique direction, preferably orthogonal, with respect to the direction identified by the axis X.

The protrusion is oriented in a radially outer direction, but such an orientation should not be intended as limiting, since in an alternative embodiment this orientation could be radially extending inwards.

During the step of assembly the deformation of part of the second body 7 allows the introduction thereof within the lateral wall 52. The extraction is operatively prevented by the shoulder 5k. For the purposes of the present disclosure, as “operatively prevented”, it is intended that the extraction of the second body 7 from the lateral wall 52, in such a way that at least the sealing element 71 is axially outside of the shoulder 5k, it is only possible through a breaking. Furthermore, the force required to do so it is preferably greater with respect to the common force available for an adult and robust human being, or at least for a human being who has no specific intention of destroying the stopper 1. Thanks to this technical feature it is possible to reduce the risk that an operator, a user or a child, may inadvertently extract the body 7 determining an unintentional opening of the tank 6 with consequent leakage of the substance 2.

### Alternative Embodiments for the First and the Second Portion

The Applicant notes that the presence, on the dividing wall 4p of the first portion, of the pair of discharge holes 9 and the presence, on the dividing wall 5p of the second portion 5, of the through holes 54 and of the closing elements 10, is not to be intended as limiting. In particular, the Applicant notes that alternative embodiments of the stopper 1 herein described, even if not shown, can provide for a configuration in this regard substantially inverted, wherein the dividing wall 5p of the second portion 5 provides for the presence of at least one, or of a plurality of, discharge holes 9, whereas the through holes and the closing elements 10, either in pairs or in larger numbers, or as a single unit, can be arranged on the dividing wall 4p of the first portion 4 of the first body 4, 5.

### The Relative Movement Between the First Portion 4 and the Second Portion 5 for the Opening of the Tank 6

During the relative movement between the first portion 4 and the second portion 5 in particular through a rotation between them around the axis X, this relative rotation determines a movement of the closing elements 10 inside the recesses 11. In order to open the draining holes 9, determining therefore the opening of the tank 6, starting from a configuration similar to the one of FIG. 10, the relative rotation between the first portion 4 and the second portion 5 of the first body 4, 5 determines a movement of the closing elements 10 from the first end of the recesses 11 towards the second end of the recesses 11. The closing elements move from the draining holes 9, if present thanks to the connection portion 11r and/or thanks to the action of bending of the dividing wall 5p, by freeing at first partially and then integrally the draining holes 9.

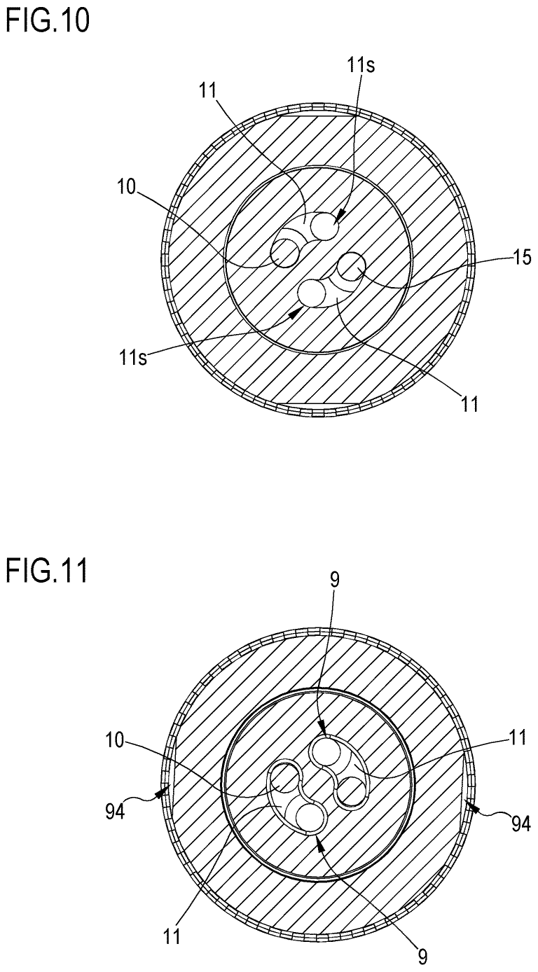

Prosecuting with the relative rotation of the first portion 4 with respect to the second portion 5 of the first body 4, 5 the closing elements 10 arrive in correspondence of the second ends of the recesses 11, striking against the wall of the recess itself. If present, at this point the closing elements 10 enter into the blind holes 11s (FIG. 11).

With the progressive movement of the first portion 4 with respect to the second portion 5 of the first body 4, 5, the through holes 54 progressively align axially to the draining holes 9 until they reach a complete overlap when the closing elements 10 are in correspondence of the second ends of the recesses 11.

In light of the above, it is therefore clear that defined as “second relative position” between the first portion 4 and the second portion 5 of the first body 4, 5 the one wherein the closing elements 10 are in correspondence of the discharge holes 9 and/or in correspondence of the first end of the recesses 11, and defined as “first relative position” between the first portion 4 and the second portion 5 of the first body 4, 5 the one wherein the closing elements 10 are in correspondence of the blind holes 11s or anyway in substantial correspondence of the second end of the recesses 11, it is clear that when the first portion 4 is in said second relative position with respect to the second portion 5, the discharge holes 9 are insulated with respect to said tank 6; when the second portion 4 is in said first relative position with respect to the second portion 5 of the first body 4, 5, the discharge holes 9 communicate with said tank 6.

In other words, when the first portion 4 is in said second relative position with respect to the second portion 5 of the first body 4, 5, the closing element 10 is positioned at least partially, preferably integrally, within the respective discharge hole 9 and/or seals said discharge hole 9; when the first portion 4 is in said first relative position with respect to the second portion 5 of the first body 4, 5, the closing element 10 is positioned at least partially, preferably integrally, out of the respective discharge hole 9 and/or frees at least partially, preferably integrally, the respective discharge hole 9.

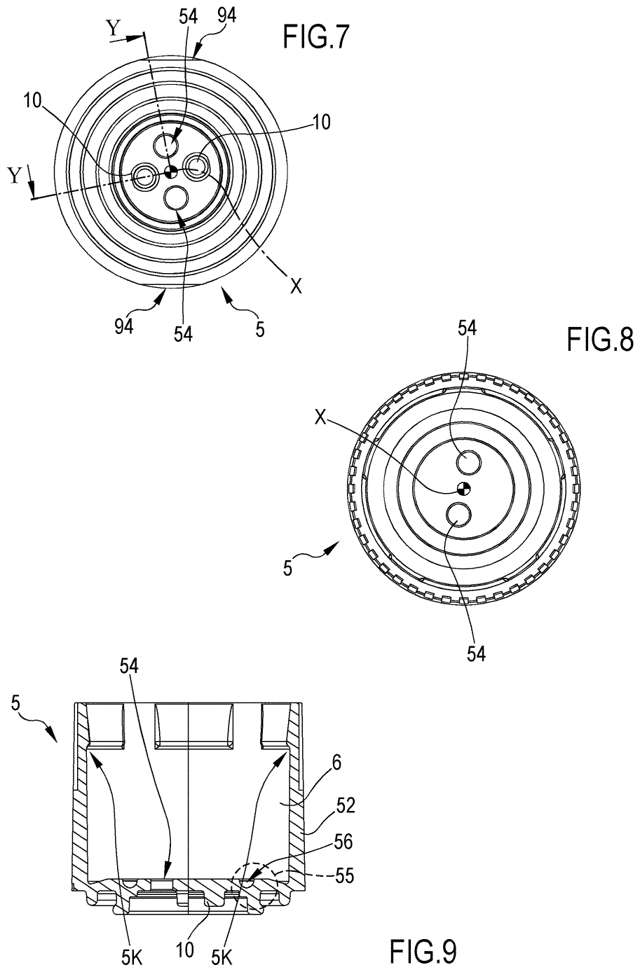

In a preferred embodiment, the stopper 1 object of the present disclosure comprises positional reference elements 94 which are configured and specifically destined to allow to identify a predefined spatial orientation of the stopper 1. Positional reference elements 94, in opposing position, are for example represented in FIG. 7 or in FIG. 10. More in particular, the spatial reference elements 94 are configured and specifically destined to allow to identify an orientation of the stopper 1 with respect to the rotation axis X.

In the embodiment of the stopper 1 represented in the attached figures, the positional reference elements 94 are arranged in correspondence of the first portion 4 of the first body 4, 5 and allow to identify a reference position of the first portion 4 of the first body 4, 5 with respect to the rotation axis X. In this way the positional reference elements 94 allow also to identify a reference position of the second portion 5 of the first body 4, 5 with respect to the rotation axis X.

Preferably, the positional reference elements 94 are notches realized on the outer surface of the first portion 4 of the first body 4, 5. This is the solution represented in the attached figures, where in particular the positional reference elements 94 in form of notches are numerically two and are arranged in an opposite way. The positional reference elements 94 in form of notches could be numerically equal to three or more, or there could also be a single positional reference 94 in form of a notch.

The notch is realized in such a way as to allow to define a first accessible outer portion of the stopper 1 and a second accessible outer portion of the stopper 1 (different from the first accessible outer portion of the stopper 1) which is non-axisymmetric with the first accessible outer portion of the stopper 1.

Alternatively, the positional reference elements 94 can be realized in form of protrusions, and identify a convexity on an outer side of the coupling wall 42.

Still alternatively, the positional reference elements 94 can be realized as local alteration of a pattern of the knurling present on the outer side of the coupling wall 42, and/or comprise a graphic (for example a QR code) or alphanumeric pattern realized through printing, painting or laser engraving. In this case the positional reference elements 94 can be substantially co-planar on the outer side of the coupling wall. The positional reference elements 94 can be arranged in substantial correspondence of the second portion 5 of the first body 4, 5, alternatively or in combination to positional reference elements 94 arranged on the first portion 4 of the first body 4, 5.

The positional reference elements 94 are conceived with the purpose of facilitating the manual, partially manual or integrally automated, assembly of the first portion 4 of the first body 4, 5 with the second portion 5, of the first body 4, 5. This assembly occurs in particular identifying a predefined relative rotation position of the first portion 4 of the first body 4, 5 with respect to the second portion 5 of the first body 4, 5 which allows an axial alignment between a closing element 10 and the respective recess 11.

The positional reference elements 94 are also conceived with the purpose of promoting a handling of the stopper 1 during the use, in particular in the steps prodromic to the release of the substance 2 by effect of the movement of the second body 7 with respect to the first body 4, 5.

### The Second Body 7

The second body 7 is at least partially introduced within the first body 4, 5, and in particular is at least partially introduced within the second portion 5 of the first body 4, 5.

Preferably, but in a non-limiting extent, the second body 7 substantially identifies a rotation solid which develops along a predefined main axis. This main axis, when the second body 7 is coupled in operating configuration with the first body 4, 5, coincides with the main development axis X of the stopper 1. In a preferred embodiment, at least taking a first and a second portion thereof, this second body 7 is substantially axisymmetric.

In a preferred but non-limiting embodiment, the second body 7 is a one-piece body, integral, preferably realized in a single injection moulding process. This guarantees a low production cost for the second body 7.

A movement, in use, of the second body 7 with respect to the first body 4, 5, in particular a movement between the second body 7 and the second portion 5 of the first body 4, 5, between a first position and a second position determines a variation of volume of the tank 6. In other words:
the tank 6 assumes a first volume when the second body 7 assumes said first position with respect to said first body 4, 5, in particular when the second body 7 assumes the first position with respect to the second portion 5 of the first body 4, 5, and
the tank 6 assumes a second volume when said second body 7 assumes said second position with respect to said first body 4, 5.

The first position is a maximum axial extension position of the stopper 1 and/or of the second body 7 with respect to the first body 4, 5, in particular with respect to the second portion 5 of the first body 4, 5. In this first position the second body 7 extends for a maximum portion outside of the first body 4, 5, in particular outside of the second portion 5 of the first body 4, 5.

The second position is a minimum axial extension position of the stopper 1 and/or of the second body 7 with respect to the first body 4, 5, in particular with respect to the second portion 5 of the first body 4, 5. In this second position the second body 7 extends for a minimum portion outside of the first body 4, 5, in particular outside of the second portion 5 of the first body 4, 5.

The volume of the tank 6 changes as follows:
the volume is maximum when the second body 7 assumes said first position with respect to the first body 4, 5, in particular with respect to the second portion 5 of the first body 4, 5;
the volume is minimum when the second body 7 assumes said second position with respect to the first body 4, 5, in particular with respect to the second portion 5 of the first body 4, 5.

Preferably, but in a non-limiting extent, the minimum volume is a volume substantially void.

In correspondence of an end portion, in particular the lower end portion, the second body 7 presents a sealing element 71 in form of annular wall which preferably but in a non-limiting extent results inclined downwards, and i.e. towards the portion of bottom of the tank 6 realized by the dividing wall 5p.

In a non-limiting embodiment, this sealing element can be realized in form of O-ring in elastic material and preferably polymeric, compatible with the substance contained in the tank 6.

The sealing element 71 is substantially flexible and/or deformable. When in form of annular wall, this annular wall when introduced within the lateral wall 52 of the second portion 5 of the first body 4, 5 is bent in order to determine a substantial and effective contact with the inner side of the lateral wall 52.

The sealing element 71 in form of annular wall defines a skirt which presents a relative elastic flexibility such as to bend to such an extent at least as to allow the introduction of the second body 7 into the lateral wall 52 of the second portion of the first body 4, 5. In particular the flexibility of the annular wall is preferably such as to allow that, with the above-described bending, a characteristic diameter substantially identified by it in rest condition, can be reduced temporarily by 10%-20%, for example and preferably by 15%, without breaking (to allow the introduction within the lateral wall 52) and then to return, substantially by elastic release, to the same original diameter. Conveniently then at least this annular wall is realized in an appropriate plastic material.

The second body 7 comprises a bottom wall 72 substantially arranged on a plane orthogonal with respect to the axis X. The bottom wall 72 identifies a central bottom portion of the second body 7 and is centered on the axis X. The radially outer end portion of the bottom wall 72 can be joined seamlessly with the sealing element 71, in particular when in form of annular wall.

The bottom wall 72 and the sealing element 71 upperly delimit the tank 6, and define an upper movable wall thereof. The volume of the tank 6 is for this reason delimited by at least one upper movable wall being part of the second body 7.

The bottom wall 72 houses preferably a convex portion 73 which presents the same form of the annular recess 56; the convex portion 73 is configured to introduce within the annular recess 56 when the second body 7 is substantially pushed towards the end stroke portion which determines the reaching of the minimum volume for the tank 6.

The second body 7 comprises also a substantially cylindrical portion 74 which realizes a substantially upper portion of the second body 7 itself. The substantially cylindrical portion 74 realizes an engagement portion with the user's fingers, and is configured to enter in direct contact with the latter in order to be moved between the first and the second position. The substantially cylindrical portion 74 comprises a first end and a second end; the first end of the cylindrical portion 74 is joined with the bottom wall 72. In an embodiment the second body 7 is realized integrally through a known moulding technique and for this reason the joining between the various portions described herein shall intended only in order to identify them for the purpose of descriptive clarity, being in fact the second body 7 realized as a piece and there being therefore no portions joined through processing following the previously described moulding.

The substantially cylindrical portion 74 comprises a substantially smooth outer face and an inner side from which radially protrude towards the center of the substantially cylindrical portion 74 a plurality of reinforcing ribs 74c which have an extension direction substantially parallel to the axis X. In correspondence of the first portion is present a centering ring 75 which radially protrudes outwards. The maximum diameter identified by the centering ring 75 is almost close to the diameter identified by the inner side of the lateral wall 52. In this way, the centering ring 75 cooperates with the sealing element 71 in the form of annular wall to maintain an axial movement of the second body 7 along the axis X. The center of the substantially cylindrical portion 74 is therefore substantially coinciding with the axis X.

The second end of the substantially cylindrical portion 74 is open so as to be able to directly access the reinforcing ribs 74c.

The Applicant notes that the second body 7 not only is movable with respect to the first body by axial (linear) translation along the axis X but is also able to rotate around the axis X. This is true where the transverse section of the lateral wall 52 of the second portion 5 of the first body 4, 5 and, consequently, the sealing element 71, are such as to identify a substantially circular shape. This solution is clearly to be intended as preferred, but is not to be considered as limiting, since for example the inner side of the lateral wall 52 and, consequently, the sealing element 71 may present a substantially polygonal-type section, although in that case some sealing issues could be observed in correspondence of the corners.

### Peelable Element

In a preferred but non-limiting embodiment, the stopper 1 can comprise at least one peelable element 80. The peelable element 80 can be conveniently realized through a sheet of flexible and/or bendable material. This sheet can be a single-layer sheet or a multi-layer sheet.

When in single layer, the sheet can be realized in plastic material or in aluminium.

When in multi-layer, the sheet preferably comprises a layer of plastic material and a layer of aluminium.

In a preferred, but non-limiting embodiment, the peelable element 80 is joined with the first body 4, 5 through one among a gluing step, a welding step (for example through ultrasounds) or a further known technique not described herein.

Preferably, but in a non-limiting extent, the peelable element 80 can be arranged in substantial correspondence of a lower edge of the first body 4, 5. This lower edge is the one that in use first enters into contact with the opening of the container 3.

The peelable element 80 can be suitably provided when the substance 2 contained in the tank 6 is toxic, corrosive, strongly acidic or basic, strongly hydrophilic, flammable, potentially explosive or anyway capable of reacting rapidly with air or liquids (for example with exothermic reactions).

Conveniently, the use of the peelable element 80 allows to retain within the stopper 1 the substance 2 even if the user inadvertently rotates the first portion 4 of the first body 4, 5 relatively to the second portion 5 of the first body 4, 5 and possibly also presses the second body 7. The peelable element 80 therefore becomes a barrier against the accidental contact with the substance, when the first and the second portion of the first body 4, 5 are inadvertently rotated between them so as to determine the opening of the tank 6; the peelable element 80 also becomes an element capable of preventing the dispersion of the substance contained in the tank 6, should the first and the second portion of the first body 4, 5 be rotated between them so as to determine the opening of the tank 6.

The peelable element 80 can be configured to be broken as a result of and by effect of the engagement of the stopper 1 on the container 3, and/or can be broken by the rod 100.

In a non-limiting embodiment, the peelable element 80 can also be provided with a tab 81 which protrudes out of the dimension overall identified by the first body 4, 5, and in particular out of the shape overall identified by the coupling wall 42, or outer lateral wall. This tab 81 is specifically conceived to facilitate the grip to the user.

The peelable element 80 is pierceable for example through the introduction of the rod 100; it is therefore not necessary its manual removal before the introduction of the rod 100. However, such manual removal is still preferable.

### Handling for the Extraction of the Substance 2

The present disclosure generally describes a method of handling of a stopper 1; this method comprises:
a first step of engagement of the first portion 4 of the first body 4, 5,
a second step of engagement of the second portion 5 of the first body 4, 5,
a first step of movement, comprising a movement of the first portion 4 with respect to the second portion 5 between the first relative position and the second relative position, the step of movement occurring after the first and second step of engagement;
a step of engagement of the second body 7;
a second step of movement, subsequent to the first step of movement, the second step of movement comprising a movement of the second body 7 with respect to the first body 4, 5 between the first position and the second position;
the second step of movement determining a leakage at least partial of the substance 2 from the tank 6 through the at least one discharge hole 9.

The stopper 1 object of the present disclosure is specifically configured to be handled by a user. Hereinafter, it is described in more detail the operation carried out by the user in order to extract the substance 2 from the stopper 1:
engage the first portion 4 of the first body 4, 5 and engage the second portion 5 of the first body 4, 5, for example with fingers of a first and second hand;
subsequently, rotate the first portion 4 of the first body 4, 5 with respect to the second portion 5 of the first body 4, 5 so as to determine a movement of the closing elements 10 within the recesses 11 and in order to free the discharge holes 9 (or where present as single, the single discharge hole 9) to allow to place the tank 6 in open configuration (commutation between closed configuration of the tank and open configuration of the tank);
subsequently, engage the second body 7;
then axially push the second body 7 so as to move it from the first position relatively assumed with respect to the first body 4, 5, in particular with respect to the second portion 5 of the first body 4, 5 towards the second portion relatively assumed with respect to the first body 4, 5, in particular with respect to the second portion 5 of the first body 4, 5, so as to determine a progressive reduction of the volume of the tank 6 and force the leakage of the substance 2 from the at least one discharge hole 9.

The rotation occurs in a predefined sense of rotation starting from a first end stroke position assumed by the first portion 4 of the first body 4, 5 with respect to the second portion 5 of the first body 4, 5. This predefined sense of rotation can for example but in a non-limiting extent be clockwise.

The rotation ends in correspondence of a second end stroke position assumed by the first portion 4 of the first body 4, 5 with respect to the second portion 5 of the first body 4, 5. The rotation in particular ends since the conformation of the recesses 11 determines, in correspondence of the second end stroke portion, a sudden increase of the force required to rotate the first portion of the first body 4, 5 with respect to the second portion of the first body 4, 5 when the closing elements 10 enter in contact with a bottom wall of the recesses 11 and introduce themselves into the blind holes 11s, if present.

The rotation determines a movement of a closing element 10 of at least one discharge hole 9 of the stopper 1 from a first position wherein said closing element 10 closes said at least one discharge hole 9 determining the closed configuration of the tank 6 to a second position wherein said closing element 10 opens the at least one discharge hole 9 determining the open configuration of said tank 6.

The linear translation of the second body 7 with respect to the first body 4, 5 determines a progressive reduction of the volume of the tank 6 and/or a progressive leakage of the substance contained in the tank 6; it ends when at least part of the second body 7 enters in contact with an end stroke element positioned in correspondence of the first body 4, 5 (optionally a bottom wall of the tank 6).

When the previously described peelable element 80 is present, it is convenient for the user to remove it before engaging the first portion 4 of the first body 4, 5 and the second portion 5 of the first body 4, 5 in order to determine its relative rotation.

In use, then, it is convenient that the removal of the peelable element 80 occurs before the relative rotation between the first portion 4 of the first body 4, 5 and the second portion 5 of the first body 4, 5 (then, before the first step of movement) but it is strongly preferable that—by function of the peelable element 80 itself—it occurs before the linear translation of the second body 7 with respect to the first body 4, 5 and then before the second step of movement.

Preferably, even if in a non-limiting extent, at least one between the first step of engagement of the first portion 4 of the first body 4, 5 and the second step of engagement of the second portion 5 of the first body 4, 5 comprises an identification of the position of the positional reference elements 94 and the movement of the first portion 4 of the first body 4, 5 with respect to the second portion 5 of the first body 4, 5 occurs by defining, for example visually, a predefined positional relationship of rotation of the first portion 4 and/or of the second portion 5 around the axis X and subsequently performing the above-mentioned movement (rotation) in order to determine the opening of the at least one discharge hole 9.

### Manipulability Through Machine

The stopper 1 object of the present disclosure can be preferably configured to be handled by a user and to be handled by an automatic machine, for example but in a non-limiting extent by a machine for the analysis of samples, preferably samples of biological and/or microbiological material.

This machine can be in particular configured to removably engage the container 3 and/or the stopper 1, in particular in correspondence of the outer side of at least one among the following elements: the second body 7, the first portion 4 of the first body 4, 5, the second portion 5 of the first body 4, 5.

In a preferred but non-limiting embodiment, this machine is configured to retain at least temporarily the stopper 1 in a specific positional relationship with respect to the container 3, in such a way that the container 3 is arranged at a lower height with respect to the stopper itself, and with the opening facing upwards. For the purposes of the present disclosure as “upwards” is intended that direction opposed with respect to a floor on which the machine itself lies in use.

Alternatively or in combination with what has been described above, the machine herein described can be a machine comprising a handler configured to engage itself in correspondence of an inner portion of the second body 7, in particular in correspondence of the reinforcing ribs 74c, or in correspondence of the second end of the substantially cylindrical portion 74, for example to push this substantially cylindrical body so as to determine an axial movement of the second body 7 with respect to the first body 4, 5 along the axis X.

In an embodiment the machine comprises at least one handler configured to determine a relative movement of the first portion 4 of the first body 4, 5 with respect to the second portion 5 of the first body 4, 5 at least between the first relative position and the second relative position and/or to determine a relative movement between the second body 7 and the first body 4, 5 between the first position and said second position.

Preferably but in a non-limiting extent, the machine comprises a handler which in use temporarily maintains the first portion 4 of the first body 4, 5 in a predefined and fixed position, and moves the second portion 5 of the first body 4, 5 to determine the above-mentioned movement thereof, in particular rotation.

The machine can also be configured to engage the first portion 4 of the first body 4, 5 through a first handler and to engage the second portion 5 of the first body 4, 5 through a second handler. The machine can be configured to actuate the first and the second handler so as to determine a relative movement between the first portion 4 of the first body 4, 5 and the second portion 5 of the first body 4, 5, in particular to determine a relative rotation thereof around an axis substantially coinciding with the axis X.

In a preferred embodiment the machine can be configured to actuate the following:
engage the first portion 4 of the first body 4, 5 through a first handler and engage the second portion 5 of the first body 4, 5 through a second handler;
subsequently, rotate the first portion 4 of the first body 4, 5 with respect to the second portion 5 of the first body 4, 5 so as to determine a movement of the closing elements 10 within the recesses 11 and in order to free the discharge holes 9 (or where present in single unit, the single discharge hole 9) to allow to place the tank 6 in open configuration (commutation between closed configuration of the tank and open configuration of the tank);
subsequently, engage, through one between the first and the second handler, and/or through a further handler, the second body 7;
then axially push the second body 7 so as to move it from its first position relatively assumed with respect to the first body 4, 5, in particular with respect to the second portion 5 of the first body 4, 5 towards the second portion relatively assumed with respect to the first body 4, 5, in particular with respect to the second portion 5 of the first body 4, 5, so as to determine a progressive reduction of the volume of the tank 6 and force the leakage of the substance 2 from the at least one discharge hole 9.

The machine herein described can also be provided with tweezers or an equivalent device configured to engage the peelable element 80 and remove it.

The machine described herein can be configured to identify the presence of at least one positional reference element 94 on the stopper 1. In particular, in this case, the handler which engages the first body 4, 5 in use engages the first body 4, 5 in such a way that at least one between the first portion 4 of the first body 4, 5 and the second portion 5 of the first body 4, 5 is arranged in a predefined positional relationship of rotation around the axis X.

In a specific embodiment, the machine herein described can be configured to engage the first portion 4 of the first body 4, 5 and/or the second portion 5 of the first body 4, 5 in a predefined positional relationship with respect to the at least one positional reference element 94 and to engage the stopper 1, and more in particular at least one between the first portion 4 of the first body 4, 5 and the second portion 5 of the first body 4, 5 in correspondence of said positional reference element.

In order to carry out the manipulability actions described in the present disclosure, the machine herein described operates in a substantially automatic way. The various components of the machine above described are preferably controlled through a control unit comprising one or more among general purpose or dedicated type processors, ASICs, FPGAs or PLCs, and can be provided with a software program comprising portions of code which—when executed by the control unit—cause the execution of the above-described handling.

The control unit determines the actuation of mechanical, electromechanical, pneumatic or hydraulic or even optical actuators for the execution of the previously described steps.

### Removable Engagement of the Stopper With a Device for Collecting Samples

In the preceding portions of description it is described that in correspondence of the lower part of the first portion 4 of the first body 4, 5 it is present a hollow 8 substantially bell-shaped wherein it is obtained a recess 8c or cavity. This cavity 8c is destined to realize a housing configured to removably retain, in use, an end portion of a rod 100.

This rod 100 is part of a device for collecting samples, preferably for collecting samples of biological and/or microbiological material, in particular destined to collect biological and/or microbiological material from a surface or from a cavity of a human being or animal and/or to carry out collections of materials present in the environment.

The device for collecting samples comprises at least one rod 100 and one swab 101.

The rod 100 herein described is a body with substantially elongated shape that in a particular embodiment is provided with a swab 101 for example but in a non-limiting extent realized in flocked material. Preferably but in a non-limiting extent, the rod 100 is realized in flexible plastic material.

The body extends along an axial direction. When the rod is introduced in the hollow 8 and is retained in the cavity 8c, the rod extends along the axis X.

In general terms, for the purposes of the present disclosure, the swab 101 can be joined with, at least partially superimposed on, realized on, said rod 100.

In a particular embodiment this swab 101 can be configured to be separated from the rod 101 in correspondence of one or more weakening portions positioned on the body of the rod 100 itself, which may also extend into the swab 101.

In an embodiment the rod 100 can be hollow and be provided, preferably in proximity of the swab 101, of at least one draining hole, in order to allow the substance 2 leaking from the at least one discharge hole 9 to enter within the rod 100 and leak in proximity of the draining hole in proximity of the swab 101.

In a non-limiting embodiment, the swab 101 can be realized in a material specifically destined to the preservation of the biological and/or microbiological material, or chemically treated to promote the preservation of the biological and/or microbiological material. For the purposes of the present disclosure, as “preservation” it is intended the maintenance, as far as possible unaltered, of the microbiological or biological properties of the collected sample; preferably the chemical treatment can be such as to allow the maintenance of the properties of the sample for two weeks or more.

### Further Considerations Regarding the Realization of the Body of the Stopper 1

The stopper 1, and preferably both the first body 4, 5 and the second body 7, is preferably realized in plastic material, resistant to acids and/or corrosive substances and/or is realized in biodegradable plastic material, suitable for not releasing substances when in contact with biological samples.

The use of a plastic material advantageously allows to realize the stopper 1 in a cost-efficient way, through for example injection moulding processes.

In a particular embodiment, the plastic material with which the stopper is realized is opaque, and therefore the substance 2 cannot be seen from the outside of the stopper itself. By hiding the substance 2, and not allowing it to be freely transferred into the container 3, the risk that determined persons, among which children, who potentially have access to the stopper 1 described herein ingest or otherwise come into contact with the substance 2 is reduced. The use of an opaque stopper is also advantageous when the substance 2 contained in the tank 6 is photosensitive, since in this way the risk of degradation of the substance 2 itself is reduced, even when the stopper object of the present disclosure should be left in areas exposed to light for a long time.

Although so far a release stopper 1 has been described as provided with threading for the coupling with the container 3, this specific technical characteristic is not to be intended as limiting; in fact, in an embodiment not shown in the attached figures, the removable coupling could be realized through clips, arranged on the stopper 1 and suitable for engaging in undercuts present on the container 3 or, vice versa, through clips arranged on the container 3 and suitable for engaging in undercuts present on the outer surface of the stopper 1. It is observed here that the clips may be configured to cause a relevant compression of the stopper 1 against the container 3 during the closing, determining a reduction of the axial extension along the axis X of the assembly formed by the stopper 1 and the container 3.

The stopper 1 object of the present disclosure can be realized and sold alone, or can also be realized and/or sold as a kit. In particular, the kit previously described may comprise the stopper 1 and at least one among:
a device 100, 101 for collecting samples, preferably for collecting biological and/or microbiological material, which as previously described comprises a rod 100 and a swab 101, joined with, at least partially superimposed on, realized on, said rod 100 and configured and specifically destined to allow the collection and/or the storage of a sample of biological and/or microbiological material;
a container 3 for samples, preferably for samples of biological and/or microbiological material, the container being configured and specifically destined to be removably coupled to the stopper 1 in correspondence of an opening, and being specifically destined to house the device 100, 101 for collecting samples;
an adapter, configured and specifically destined to be removably coupled to the stopper 1 in correspondence of the first body 4, 5, optionally in correspondence of the first portion 4 of the first body 4, 5; said adapter being configured and specifically destined to removably couple the stopper 1 to an auxiliary device.

For example, and in a non-limiting extent, this auxiliary device can be an auxiliary container for samples, or a duct for the release of the substance 2, or even a cartridge.

The adapter is configured and specifically destined to be removably coupled to the first body, in particular to the first portion 4 of the first body 4, 5. Preferably, but in a non-limiting extent, the adapter is provided with a head wall provided with threading, in particular with a threading compatible with the threading arranged on the coupling wall 42.

In a preferred, but non-limiting embodiment, the adapter is realized in plastic material, for example in the same plastic material as it is realized one between the first body 4, 5 or the second body 7. This allows to slightly optimize the production costs of the kit.

The object of the present disclosure is not limited to the embodiments represented in figures; for this reason, numbers and reference marks in the claims are to be considered non-limiting. They are provided for the sole purpose of increasing the intelligibility of the claims.

It is finally clear that modifications or variants obvious for a technician of the art may be applied to the object of the invention without departing from the scope provided by the attached claims.

1. A stopper for releasing a substance into a container for samples, comprising at least:
a first body configured to be removably engaged on said container,
a second body coupled and movable with respect to the first body,
at least one tank configured to contain the substance and defined in the first body or between the first body and the second body;
wherein the first body comprises at least:
a first portion configured to be directly coupled on said container and comprising a coupling element configured to allow the removable fastening of the stopper in correspondence of an opening of the container defining a first mounting configuration of the stopper on the container wherein the stopper closes the container,
a second portion movably connected with said first portion, said second portion realizing at least part of a bottom wall and/or of a lateral wall of said tank, said second portion being capable to lie in at least one first relative position with respect to said first portion and in at least one second relative position with respect to said first portion;
said second portion of the first body being directly coupled to said second body,
the tank having at least one first open configuration and one second closed configuration, said first open configuration being determined by the first relative position between the first portion of the first body and the second portion of the first body, said second closed configuration being determined by the second relative position between the first portion of the first body and the second portion of the first body.

2. The stopper according to claim 1,
wherein the tank is a tank having a volume variable at least between a first volume and a second volume, the second volume being smaller with respect to the first volume,
wherein the second body is configured to assume at least one first position and one second position with respect to said first body,
and wherein a movement, in use, of the second body with respect to the first body between said first position and said second position determines a variation of volume of said tank and/or wherein the tank assumes the first volume when said second body is in said first position with respect to said first body and wherein the tank assumes the second volume when said second body is in said second position with respect to said first body;
wherein the second body is movable with respect to the first body between said first position and said second position through at least one axial translation;
wherein said first position and said second position are end stroke positions for said axial translation, and wherein said first volume is a maximum volume and/or wherein said second volume is a minimum volume;
the stopper presenting a main development axis and said axial translation occurring along a direction, parallel to said main development axis.

3. The stopper according to claim 1, comprising at least one discharge hole configured to discharge the substance from said tank,
wherein said at least one discharge hole is arranged in said first portion of the first body,
and wherein said second portion of the first body comprises at least one closing element configured to removably engage within said at least one discharge hole and comprises at least one through hole configured to couple with said discharge hole alternatively to said at least one closing element, or wherein said at least one discharge hole is arranged in said second portion of the first body and wherein said first portion of the first body comprises at least one closing element configured to removably engage within said at least one discharge hole and comprises at least one through hole configured to couple with said discharge hole alternatively to said closing element,
wherein when the first portion of the first body is in said second relative position with respect to the second portion of the first body, the closing element is positioned at least partially within said discharge hole and/or seals said discharge hole and the through hole is uncoupled with respect to said discharge hole, and wherein when the first portion of the first body is in said first relative position with respect to the second portion the closing element is positioned at least partially out of said discharge hole and/or frees at least partially said discharge hole, and the through hole is coupled to said discharge hole,
wherein when the first portion of the first body is in said second relative position with respect to the second portion of the first body, said at least one discharge hole is sealed with respect to said tank and wherein when the second portion of the first body is in said first relative position with respect to the second portion of the first body, said at least one discharge hole communicates with said tank.

4. The stopper according to claim 1, configured to be removably coupled with a rod of a device for collecting samples of biological or microbiological material,
wherein the first portion of the first body comprises at least one cavity realizing a housing configured to removably retain, in use, an end portion of said rod,
the first portion of the first body comprising at least one channel, communicating with said at least one discharge hole, said at least one channel being configured and specifically destined to allow an evacuation of the substance extracted from the tank in substantial proximity of the rod.

5. The stopper according to claim 4, wherein the cavity is configured to allow an introduction of the end portion of said rod through an axial insertion,
the cavity being configured to allow an axial insertion of the end portion of said rod along an axis parallel or coinciding with a main development axis of said stopper,
the second portion of the first body comprising, in correspondence of said cavity, at least one retaining rib,
said at least one retaining rib radially protruding towards a center of said cavity, and being destined to exert a sliding friction with said end portion of said rod when introduced within said cavity;
the at least one retaining rib being elastically deformable.

6. The stopper according claim 1, wherein the second portion of the first body is rotatably connected with respect to said first portion of the first body and wherein between said first relative position and said second relative position there is a predetermined rotation between said first portion of the first body and said second portion of the first body,
wherein the second portion of the first body is rotatably connected with respect to said first portion of the first body and is configured to rotate with respect to said first portion of the first body and on an axis substantially coinciding with a main development axis of said stopper and/or wherein the second portion of the first body is rotatably connected with respect to said first portion of the first body on an axis substantially coinciding with a translation axis of the second body with respect to the first body, the translation axis of the second body with respect to the first body being substantially coinciding with the main development axis of said stopper.

7. The stopper according to claim 3, wherein at least one between said first portion of the first body and/or said second portion of the first body comprises a respective dividing wall which is deformable and wherein said dividing wall is at least partially arranged in an orthogonal direction with respect to the main development axis and/or is arranged in an orthogonal direction with respect to the translation axis of the second body with respect to the first body;
and wherein a movement between the first portion of the first body and the second portion of the first body between said first relative position and said second relative position determines, in use, a deformation of at least one between the dividing wall of the first portion and the dividing wall of the second portion of the first body;
the dividing wall of the second portion of the first body realizing a bottom wall of said tank.

8. The stopper according to claim 3, wherein alternatively the at least one discharge hole is realized on the dividing wall of the first portion of the first body and the at least one through hole and the at least one closing element are arranged on the dividing wall of the second portion of the first body or wherein the at least one discharge hole is realized on the dividing wall of the second portion of the first body and the at least one through hole and the at least one closing element are arranged on the dividing wall of the first portion of the first body.

9. The stopper according to claim 1, wherein the first portion of the first body, comprises at least one first recess within which slides the at least one closing element during a relative movement between the first portion and the second portion of the first body;
said recess being a recess substantially identifying a curved profile, and/or housing the at least one discharge hole;
wherein the at least one discharge hole lies in substantial correspondence of a first end of the at least one first recess;
the at least one first recess being arranged on said dividing wall of the first portion of the first body.

10. The stopper according to claim 1, configured to allow a movement from the first relative position toward the second relative position between the first portion of the first body and the second portion of the first body through an initial snap and/or through an initial imposition of force greater with respect to a force required to complete the movement from said first relative position toward said second relative position.

11. The stopper according to claim 3, wherein an extraction of said at least one closing element from said at least one discharge hole is made possible by said initial snap and/or by means of said initial imposition of force greater with respect to a force required to complete the movement from said first relative position to said second relative position.

12. The stopper according to claim 8, wherein said at least one first recess has a substantially elongated shape and/or has at least one main development direction, and defines a first end stroke position and a second end stroke position for the relative movement, preferably the relative rotation, between the first portion and the second portion of the first body.

13. The stopper according to claim 1, comprising at least one safety or engagement element, configured to allow a movement of the first portion of the first body with respect to said second portion of the first body from said first relative position toward said second relative position and to prevent a movement, in particular a backward movement, from said second relative position to said first relative position;
said safety or engagement element being configured to allow a rotation of the second portion of the first body with respect to the first portion of the first body in a first sense of rotation and to prevent, upon reaching a predefined position of rotation between said second portion of the first body and said first portion of the first body, a rotation of said second portion of the first body with respect to the first portion of the first body in a second sense of rotation opposed with respect to said first sense of rotation.

14. (canceled)

15. The stopper according to claim 1, wherein the second portion of the first body comprises a lateral wall delimiting at least part of said tank, and wherein the second body is at least partially introduced in the second portion of the first body and contacts said lateral wall;
the second body comprising a sealing element in use in direct contact with said lateral wall; said sealing element being deformable, in particular elastically deformable, and being deformed when the second body is at least partially introduced in the second portion of the first body and contacts said lateral wall;
the second body comprising a bottom wall substantially joined with said sealing element, the bottom wall and the sealing element upwardly delimiting the tank
optionally wherein the sealing element comprises an annular wall radially arranged in an outer position of the bottom wall or radially protruding from said bottom wall; said annular wall comprising a radially outer portion exerting a friction, in particular a sliding friction, during the movement of the second body between the first and the second position.

16. The stopper according to claim 1, comprising a peelable element arranged in substantial correspondence of the first portion of the first body; the peelable element being configured and specifically destined to act as a barrier from the contact or against an extraction of said substance when leaking from the tank through the at least one discharge hole;
the peelable element comprising a sheet of flexible and/or bendable material;
the peelable element being in use united to said first portion of the first body through at least one between a gluing process and/or a welding process.

17. The stopper according to claim 1, wherein the second portion of the first body comprises an engagement portion with the first portion of the first body, the engagement portion being configured to maintain movably constrained one end of the second portion;
the engagement portion being configured and specifically destined to determine and maintain a centering of the second portion on the first portion. the

18. A kit, comprising:
a stopper according to claim 1,
and at least one among:
a device for collecting samples of biological or microbiological material, comprising a rod and a swab, joined with, at least partially superimposed on, realized on, said rod, and configured to allow the collection and/or storage of a sample, of biological or microbiological material;
a container for samples of biological or microbiological material, the container being configured and specifically destined to be removably coupled to said stopper in correspondence of an opening and being specifically destined to house said device for collecting samples;
an adapter, configured and specifically destined to be removably coupled to the stopper in correspondence of the first body; said adapter being configured and specifically destined to removably couple the stopper to an auxiliary device, said at least one auxiliary device comprising at least one among an auxiliary container for samples, a duct for the release of the substance, a cartridge.

19. A method of handling of a stopper according to claim 1, comprising:
a first step of engagement of the first portion of the first body,
a second step of engagement of the second portion of the first body,
a first step of movement, comprising a movement of the first portion with respect to the second portion between said first relative position and said second relative position, the first step of movement occurring after the first and the second step of engagement;
a step of engagement of the second body;
a second step of movement, subsequent to the first step of movement, the second step of movement comprising a movement of the second body with respect to the first body between said first position and said second position;
the second step of movement determining a leakage at least partial of the substance from the tank through at least one discharge hole;
the method of handling being at least partially automated and being carried out by a machine for handling stoppers for releasing a substance into a container for samples.

20. The method according to claim 19, wherein the first step of movement comprises a rotation of the first portion with respect to the second portion between said first relative position and said second relative position and/or wherein the second step of movement comprises a linear translation of the second body with respect to the first body, taking place along a direction substantially parallel to a main development axis of the stopper,
and wherein said rotation occurs in a predefined sense of rotation starting from a first end stroke position assumed by the first portion of the first body with respect to the second portion of the first body;
said rotation ending in correspondence of a second end stroke position assumed by the first portion of the first body with respect to the second portion of the first body;
said rotation determining a movement of a closing element of at least one discharge hole of the stopper from a first position wherein said closing element closes said at least one discharge hole determining a closed configuration of said tank to a second position wherein said closing element opens said at least one discharge hole determining an open configuration of said tank.

21. The stopper according to claim 17, wherein the engagement portion is configured and specifically destined to determine and maintain a centering of the movement of the second portion with respect to the first portion between said first relative position and said second relative position.
